(19) United States
(12) Patent Application Publication
(10) Pub. No.: US 2026/0053572 A1
(43) Pub. Date: Feb. 26, 2026

(54) THORACIC ACCESS TUNNELING TOOL WITH LIGHT EMITTER FOR VISIBILITY

(71) Applicant: Medtronic, Inc., Minneapolis, MN (US)

(72) Inventors: Matthew J. Hoffman, St. Paul, MN (US); Vladimir P. Nikolski, San Diego, CA (US); Bridget A. Cavanagh, White Bear Township, MN (US); Claire A. Winchester, Columbia Heights, MN (US); Hongyong Li, Spokane Valley, WA (US)

(21) Appl. No.: 19/305,054

(22) Filed: Aug. 20, 2025

Related U.S. Application Data
(60) Provisional application No. 63/685,592, filed on Aug. 21, 2024.

(51) Int. Cl. A61B 34/20; A61B 1/00; A61B 1/04; A61B 1/06; A61B 1/07; A61B 17/32

(52) U.S. Cl. CPC A61B 34/20; A61B 1/00045; A61B 1/00066; A61B 1/04; A61B 1/0655; A61B 17/320016; A61B 1/063; A61B 1/0684; A61B 1/07; A61B 2017/320056; A61B 2034/2055

(57) ABSTRACT

A tunneling tool includes a handle, a tunneling shaft extending from the handle and defining a length, and one or more lighting elements disposed along the length of the tunneling shaft, each of the one or more lighting elements configured to emit light detectable through tissue of a patient by an optical sensor.

### RELATED APPLICATIONS

This application claims the benefit of U.S. Provisional Application Ser. No. 63/685,592, filed Aug. 21, 2024, the entire contents of each of which are incorporated herein by reference.

### TECHNICAL FIELD

The present disclosure relates to tunneling tools and, more particularly, to tunneling tools for thoracic access.

### BACKGROUND

Some medical procedures may include crossing multiple tissue layers to gain access to a location within the body of a patient. Such medical procedures may include implanting one or more medical devices or components thereof, e.g., medical electrical leads, at the location. One manner of accessing an intrathoracic location is substernally and includes traversing one or more layers of tissue, e.g., diaphragmatic attachments that attach the diaphragm to the sternum. An example of a procedure is the implantation of the distal portions of one or more leads substernally, and may include using an implant tool to access the intrathoracic cavity of the patient. The one or more leads may be part of an implantable cardiac system (e.g., implantable cardioverter-defibrillator (ICD)) that may be used to deliver electrical stimulation pulses to the patient's heart to terminate cardiac arrhythmias, such as ventricular fibrillation, bradycardia, or the like. Such implantable cardiac systems may include, or may be part of a system that includes, a subcutaneously-implantable housing that encloses a pulse generator or other electronics. The housing of some cardiac systems may be connected to the one or more leads, which may be configured to deliver defibrillation and/or pacing pulses.

In order to gain access to the heart for various heart-related procedures, a physician typically performs a sternotomy. A sternotomy is a surgical procedure that allows a doctor to reach the heart (or nearby organs) and blood vessels by first making an incision in the skin over the breastplate or sternum and then cutting through the sternum. A rib spreader may be utilized to expand the ribcage to gain internal access to the organs. When surgery is finished, the doctor repositions the sternum and uses a combination of staples and wire to secure the sternum in place while the patient heals. Over time, scar tissue and adhesions form to maintain the sternum in place as the patient returns to normal life. Surgeons may be hesitant to perform procedures requiring substernal access, such as those required to implant portions of an implantable cardiac stimulation system intrathoracically, on patients that have had a previous sternotomy.

### SUMMARY

This disclosure provides tools and implant techniques utilizing such tools to gain access to spaces within a patient, such as an anterior mediastinum or other extravascular thoracic spaces, e.g., to facilitate implantation of medical devices or components thereof, such as a lead, within the space. The tools may include one or more features that provide advantages, e.g., with respect to safety, during such procedures. These features may be particularly advantageous in the case of patients that have had a previous sternotomy but require intrathoracic implantation of medical devices or components thereof. For example, the tools may include one or more lighting elements and one or more optical components to facilitate visualization during the procedure.

In some examples, a tunneling tool includes: a handle; a tunneling shaft extending from the handle and defining a length; and one or more lighting elements disposed along the length of the tunneling shaft, each of the one or more lighting elements configured to emit light detectable through tissue of a patient by an optical sensor.

In some examples, an implant system includes: a tunneling tool including: a handle comprising an activation element; and a tunneling shaft extending from the handle and defining a length; a plurality of lighting elements disposed along the length of the tunneling shaft, wherein each of the plurality of lighting elements is configured to emit light at a different wavelength or flash light at different time intervals; and an endoscope comprising a camera configured to visualize a dissection path of the tunneling tool through visualization of light emitted by the plurality of lighting elements through tissue of the patient using the camera.

In some examples, a method includes: inserting an endoscope into a patient, wherein the endoscope includes a camera; inserting a tunneling shaft of a tunneling tool into a substernal space of a patient, wherein the tunneling tool includes: a handle; the tunneling shaft extending from the handle and defining a length; and one or more lighting elements disposed along the length of the tunneling shaft; actuating an activation element configured to activate the one or more lighting elements in response to actuation of the activation element; and visualizing a dissection path of the tunneling tool through visualization of light emitted by the one or more lighting elements using the camera.

This summary is intended to provide an overview of the subject matter described in this disclosure. It is not intended to provide an exclusive or exhaustive explanation of the systems, devices, and methods described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF DRAWINGS

The following drawings are illustrative of example embodiments and do not limit the scope of the invention. The drawings are not to scale (unless so stated) and are intended for use in conjunction with the explanations in the following detailed description. Examples will hereinafter be described in conjunction with the appended drawings wherein like numerals denote like elements.

FIGS. 1A-B are conceptual diagrams illustrating an exemplary extravascular implant.

FIG. 2 is a conceptual diagram illustrating a sub-sternal access.

FIGS. 3-4 are conceptual diagrams illustrating an example tunneling device in accordance with the present disclosure.

FIG. 5 is a conceptual diagram illustrating an example implant system in accordance with the present disclosure.

FIG. 6 is a conceptual diagram illustrating an example computing device of the system of FIG. 5.

FIGS. 7A-7B are conceptual diagrams illustrating example tunneling devices including example lighting elements in accordance with the present disclosure.

FIGS. 8-11 are conceptual diagrams illustrating example lighting elements with respect to cross sections of example tunneling shafts in accordance with the present disclosure.

FIG. 12 is a conceptual diagram of an example introducer sheath that may be used in conjunction with an example tunneling device in accordance with the present disclosure.

FIGS. 13A-13C are conceptual diagrams illustrating example arrangements of a lighting element with respect to an example introducer sheath and example tunneling shaft in accordance with the present disclosure.

FIG. 14 is a conceptual diagram of an example distal portion of an example tunneling shaft in accordance with the present disclosure.

FIGS. 15A and 15B are conceptual diagrams of example distal portions of an example tunneling shaft in accordance with the present disclosure.

FIG. 16 is a flow diagram of an example technique for operating an implant system in accordance with the present disclosure.

The details of one or more examples of this disclosure are set forth in the accompanying drawings and the description below. Other features, objects, and advantages of this disclosure will be apparent from the description and drawings, and from the claims.

### DETAILED DESCRIPTION

The following detailed description is exemplary in nature and is not intended to limit, in any way, the scope, applicability, or configuration of the tools and techniques described in this disclosure. Rather, the following description provides practical examples, and those skilled in the art will recognize that some of the examples may have suitable alternatives.

Twenty to thirty percent of patients who are otherwise eligible for ICD implantation have had prior sternotomies. A prior sternotomy is a contraindication for typical single-chamber extravascular ICD implantation procedures. A modified implantation procedure allows for more lateral implantation of an ICD from outside the sternocostal border, reducing the risk of adverse effects for patients with prior sternotomies. However, careful trajectory of the tunneling tool is crucial in such a procedure to avoid damaging thoracic tissues, such as the heart, lungs or blood vessels that feed skeletal muscles within the ribs and the thoracic area. In order to ensure safe trajectory of implantation tools and proper implantation, direct visualization of the tools during implantation is highly beneficial. Existing solutions may incorporate a camera within a tunneling shaft (e.g., a tip thereof), however, a camera incorporated within the tunneling shaft has a very narrow view of internal patient anatomy, and cannot visualize the profile of the tunneling tool itself within patient anatomy. A separate endoscope may be used to visualize the tunneling tool during the implant procedure in a video assisted thoracotomy surgery (VATS) procedure, however, the tunneling tool may be difficult to see through patient tissue from an endoscope positioned in the pleural cavity.

This disclosure describes a tunneling tool with one or more optical components incorporated in the shaft of the tunneling tool to assist visualization of the tunneling tool during an implantation procedure. For example, one or more lighting elements disposed on or within the tunneling shaft may emit light that passes through the patient tissue, illuminating a position of the tunneling shaft in the patient. The emitted light may be visible to an optical device (e.g., the camera of an endoscope) disposed in the pleural cavity of the patient. In some cases, the one or more lighting elements may be located along a length of the tunneling shaft at key features of the tunneling shaft or at locations along the length corresponding to features of a lead after implantation.

Processing circuitry may change an operation state of the one or more lighting elements in response to one or more predefined criteria being met. For example, the handle of the tunneling tool may include a trigger that, when actuated, causes control circuitry to activate the one or more lighting elements incorporated with the tunneling shaft. In some examples, successive actuations of the trigger may cause the control circuitry to change the way the one or more lighting elements emit light, e.g., from emitting continuous light to flashing light at particular time intervals. In some examples, the tunneling tool may include a pressure sensor configured to sense a pressure inside the patient. In some examples, the pressure sensor may measure a force with which part of the tunneling tool is pressing against patient tissue. The control circuitry may be configured to change the one or more lighting elements from a continuous state to a flashing state in response to the sensed pressure satisfying a threshold. In some examples, each lighting element of the one or more lighting elements may emit light at a different wavelength or flash at different time intervals.

The tunneling tool according to this disclosure may enable safe placing of extravascular and intrathoracic lead, e.g., for ICDs, even in patients with previous sternotomies. The one or more lighting elements, especially when strategically placed as disclosed herein, may allow for easy identification of the trajectory, depth, orientation, and location of the tunneling tool with respect to patient tissues.

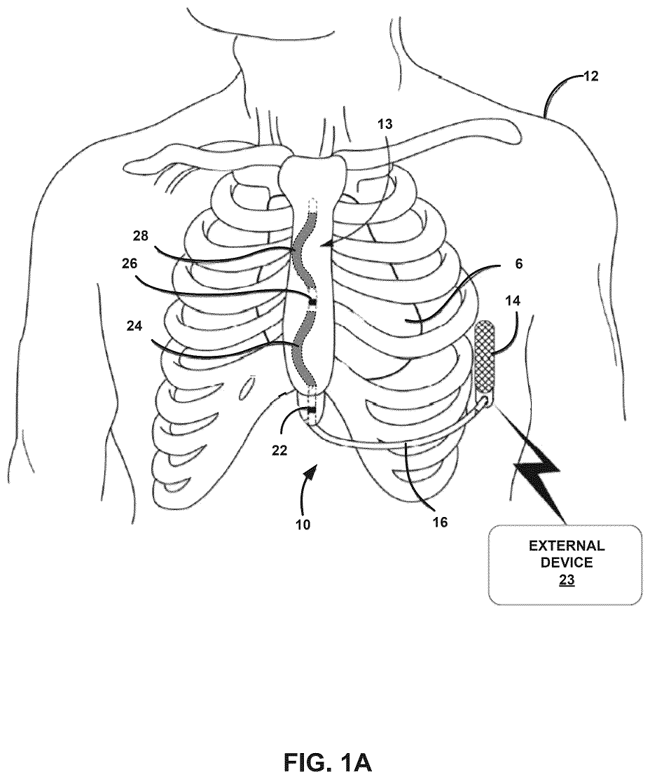

FIGS. 1A-B are schematics showing an exemplary extravascular implant of an exemplary system 10 that includes a pulse generator 14 and an implantable medical electrical lead 16 coupled thereto. Pulse generator 14 is shown implanted subcutaneously on the left mid-axillary of a patient 12, superficially of the patient's ribcage. Pulse generator 14, which may be configured to provide cardiac pacing and/or defibrillation therapy, includes a hermetically sealed housing in which the appropriate electronics and a power supply are contained, and which is formed from a conductive material, such as titanium, or from a combination of conductive and non-conductive materials. Pulse generator 14 may be in wireless communication with an external device 23 (e.g., a computing device for use by a patient, a clinician, etc.) to transmit information to external device 23, be programmed by external device 23, etc. Pulse generator 14 further includes a connector module by which lead 16 is electrically coupled to the electronics contained therein, for example, by electrical contacts contained within the connector module and a corresponding hermetically sealed feedthrough assembly, such as is known in the art. The conductive material of device housing may be employed as an electrode, for example, to provide the aforementioned therapy in conjunction with one or more pace/sense electrodes 22, 26 and/or defibrillation electrodes 24, 28 of lead 16, which is shown implanted in a sub-sternal space 3, for example, within the loose connective tissue and/or sub-sternal musculature of the anterior mediastinum. Although generally described with respect to sub-sternal space, the apparatus and techniques of this disclosure may be utilized to tunnel to the anterior mediastinum or other intrathoracic locations that need not necessarily be substernal.

Lead 16 may have any of a number of configurations. For example, lead 16 may include more or fewer pace/sense electrodes. In another example, lead 16 may include more or fewer than two defibrillation electrodes 24, 28 and/or have a defibrillation electrode(s) that is formed of multiple segments. Examples of leads with multiple defibrillation electrodes and/or segments are described in commonly assigned, co-pending U.S. Patent Publication No. 2015/0306375 (Marshall et al.), U.S. Patent Publication No. 2015/0306410 (Marshall et al.) and U.S. Patent Publication No. 2016/0158567 (Marshall et al.), each of which is incorporated herein by reference in its entirety.

With reference to FIG. 1B, sub-sternal space 3 may be viewed as being bounded laterally by pleurac 39 that form pleural cavities 35 enclosing the patient's lungs, posteriorly by the pericardial sac 15 that encloses the patient's heart 6, and anteriorly by a sternum 13. In some instances, the anterior wall of the anterior mediastinum may also be formed by the transversus thoracis and one or more costal cartilages. In some examples, lead 16 may be implanted within the transverse thoracic muscles of patient 12. Although FIGS. 1A and 1B are described in the context of the distal portion of lead 16 being placed within sub-sternal space 3, in other examples, the tools and implant techniques described herein may be used to implant a distal portion of lead 16 at other locations outside the heart. In one example, the tools may be used to place the distal portion of lead 16 intra-pericardially via a percutaneous subxiphoid approach. In some examples, the tools and implant techniques described herein could be used for implanting other medical devices or components thereof and/or for other spaces within the patient, such as, implanting a leadless pacemaker on or near outside of heart via substernal access.

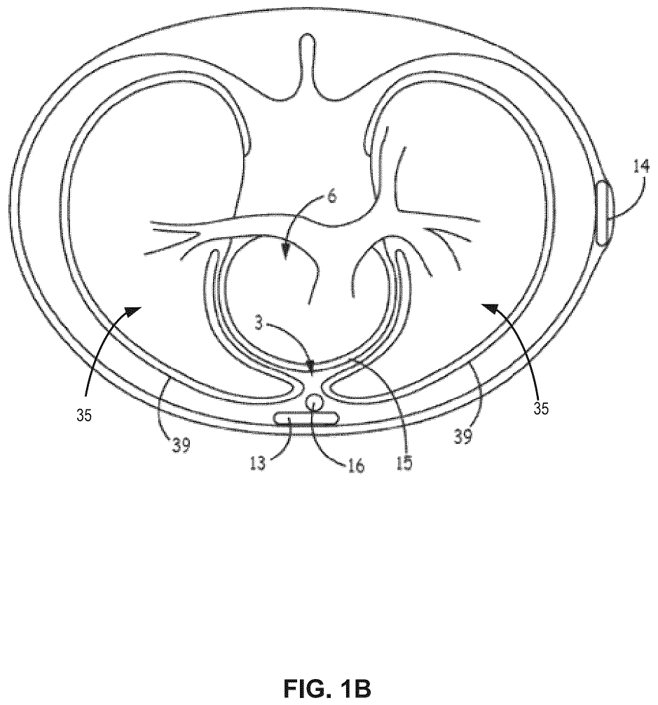

FIG. 2 is a schematic showing an access site A for making a passageway between a patient's diaphragm 19 and xiphoid process 20 of sternum 13, for example, to create a sub-sternal tunnel in which to position a medical device, such as medical electrical lead 16. After making a superficial incision, an operator, using a tunneling tool, may open a passageway between diaphragmatic attachments 18 and diaphragm 19, for example, by blunt and/or sharp dissection, in which the operator may employ a tunneling tool, such as those example tools described herein, to both create the passageway and then form a sub-sternal tunnel (e.g. along the dotted line of FIG. 2). Because the bony structure of sternum 13 inhibits external palpation, the operator may need to take extra care, during the dissection (e.g., blunt and/or sharp) and/or tunneling, not to injure sub-sternal structures or the chest cavity, which could compromise the pleura of the lungs or heart 6. Tools and associated methods disclosed herein are configured to help an operator gain the desired sub-sternal access and create a space in which to position a medical device, such as medical electrical lead 16, in a controlled fashion that mitigates the risk of injuring bodily organs.

In some examples, a distal portion of lead 16 may be implanted to be located between the posterior aspect of sternum 13 and the anterior wall of heart 6. The implant procedure may be performed by using a blunt trocar with a flexible port to create a small tunnel near the posterior aspect of sternum 13 via entry into the body near xiphoid process 20. The distal portion of lead 16, e.g., the portion of lead 16 carrying some or all of electrodes 22, 24, 26, 28, is then placed in the anterior mediastinum. The proximal end of lead 16 is then tunneled subcutaneously or submuscularly to a left midaxillary location under and connected to the pulse generator 14. Pulse generator 14 and lead 16 are able to provide, e.g., defibrillation, anti-tachycardia pacing (ATP), bradycardia pacing, post-shock pacing, and asystole “lifeboat pacing.”

Suitable tunneling tools with blunt trocars may be utilized to implant lead 16 in patient 12. However, patients that have had previous median sternotomies tend to have extensive scar tissue and the pericardium and/or anterior epicardium is often adhered to the posterior of the sternum. This scar tissue makes it very challenging and, in some cases, relatively undesirable for a blunt trocar to create a small tunnel in the anterior mediastinum. A surgeon may not want to enter the substernal space of such patients unless they have direct visualization of the location. Thus, patients that have had one or more previous median sternotomies may be less likely to receive an extravascular ICD or other medical device that includes the implantation of distal portion of lead 16 between the posterior aspect of sternum 13 and the anterior wall of the heart 6 in the manner described above.

In accordance with some examples of the disclosure, a tunneling tool (or trocar) is described that incorporates one or more lighting elements along a length of the tunneling shaft of the tunneling tool to enable visualization of the tunneling tool from an optical device external of the tunneling tool. In some examples, the optical device may be part of an endoscope used in a VATS procedure, where the endoscope is disposed within a pleural cavity of pleural cavities 35. Because of the body tissue between intended the implant location of lead 16 and the endoscope, the tunneling tool may not normally be visible by the endoscope while tunneling. The one or more lighting elements incorporated with the tunneling shaft may allow the optical device on the endoscope to visualize a trajectory of the tunneling tool during an implant procedure. Additionally, the one or more lighting elements may illuminate patient tissue structures (e.g., a cardiac silhouette, adhesions in tissue, arteries, and veins), bleeding spots, or other conditions inside the patient during the implant procedure. Such visualization may provide for better guidance of the tunneling tool during an implant procedure.

Such a tunneling tool enables safe placing of extravascular ICDs even in patients with previous sternotomies. The one or more lighting elements, especially when strategically placed as disclosed herein, may allow for easy identification of the trajectory, depth, orientation, and location of the tunneling tool with respect to patient tissues.

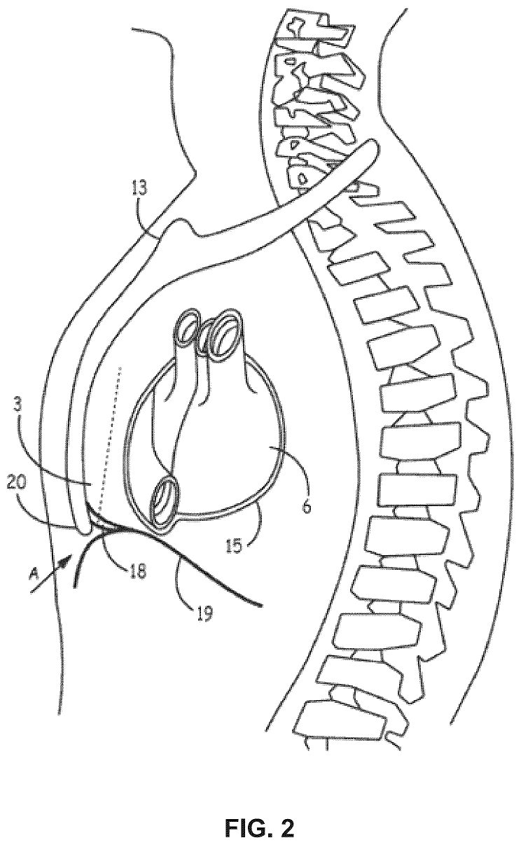

FIGS. 3-4 are functional schematic diagrams illustrating an example tunneling device 30 (also, “tunneling tool 30”) for gaining sub-sternal access and creating a sub-sternal tunnel in a patient, according to some examples. FIG. 3 illustrates tunneling tool 30 including a tunneling shaft 32 and a handle 34. FIG. 4 illustrates a cross-section view of tunneling shaft 32 about cross-section A-A shown in FIG. 3.

As shown in FIG. 3, tunneling shaft 32 of tunneling tool 30 extends from proximal end 36 to distal end 38 (or “distal tip 38”). Tunneling tool 30 also includes handle 34, which is shown coupled to proximal end 36 of tunneling shaft 32. Tunneling shaft 32 may extend from proximal end 36 to distal end 38 in a linear or straight manner. In some examples, as shown in FIG. 3, at least a portion of tunneling shaft 32 extends in a curved orientation from proximal end 36 to distal end 38, e.g., relative to an axis 40. Axis 40 may be defined by a central longitudinal axis of handle 34 or may be defined by a portion of shaft 32 that extends initially from handle 34 in a substantially straight manner before exhibiting a curved orientation beginning at a point between proximal end 36 and distal end 38 of shaft 32. The curved orientation of tunneling shaft 32 results in offset 32A between distal end 38 and axis 40 shown in FIG. 3. Offset 32A may range from approximately 0.35 inches to approximately 1.25 inches, such as, approximately 0.720 inches, although other examples are contemplated. In some instances, the curvature of tunneling shaft 32 may maintain the path of the distal tip close to posterior side of sternum and away from vital organs like lung or heart during a tunneling procedure.

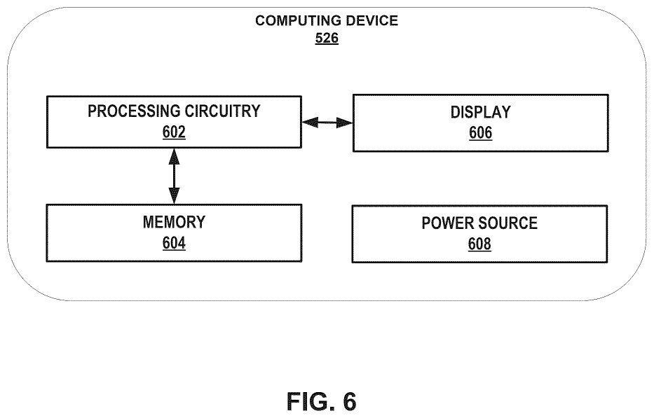

In some examples, tunneling shaft 32 may be curved about the entire length from proximal end 36 to distal end 38 (e.g., as shown in FIG. 3) or may include one or more sections that are substantially straight with one or more other sections that are curved (e.g., as shown in FIG. 7). For example, a proximate portion of the tunneling shaft 32 extending directly from handle 34 may be approximately straight for some of the length of tunneling shaft 32 and then transition to a more distal portion of tunneling shaft 32 that is curved. In some examples, the curved portion of tunneling shaft exhibits a radius of curvature of about 15 inches to about 40 inches.

Tunneling shaft 32 may be tubular, e.g., have a circular or oval outer profile and/or define one or more inner lumens, as shown in FIG. 4. Any suitable material may be used for tunneling shaft 32, e.g., metals (stainless steel, coated steel, titanium alloys, aluminum alloys and others) and plastics (unfilled and filled with suitable fiber like glass or carbon for strength and rigidity) may be utilized. Suitable plastic materials include but are not limited to acetal copolymer, polytetrafluoroethylene (PTFE) (e.g., TEFLON), polyether ether ketone (PEEK), polyphenylsulfone (PPSU) (e.g., RADEL), and polycarbonate. In some examples, tunneling shaft 32 may be formed of a material that allows for all or at least a portion of tunneling shaft 32 to be transparent along the length of shaft 32. Shaft 32 may be substantially rigid so the clinician can control accurately the position of the tip in relation to vital organs under visualization afforded by fluoroscopy or other techniques, e.g., direct visualization via an optical device incorporated within the shaft 32 or on a separate device (e.g., an endoscope). Shaft 32 may also be substantially malleable to allow a clinician to control the contour of shaft 32 before or during insertion. In some examples, the tunneling shaft 32 includes at least some metal components which will allow visualization using medical imaging technology.

In some examples, an operator may apply a force to keep tunneling shaft 32 (e.g., distal end 38) pressed against sternum 13 or ribs of patient 12. Another example force that may act on shaft 32 is a torque on shaft 32 when an operator is trying to keep shaft 32 aligned during insertion. If the operator is rotating shaft 32 back and forth along the axis 40, shaft 32 must have substantial rigidity to sweep back and forth on the posterior side of sternum 13, and clear away adhesions.

Tunneling shaft 32 may exhibit any suitable shape and dimensions. While FIG. 4 shows that tunneling shaft 32 has a substantially circular cross-section, other example cross-section shapes are contemplated. For example, tunneling shaft 32 may exhibit an oval cross-section. The outer diameter (in the case of a circular cross-section) or greatest outer dimension (in the case of a non-circular cross-section) of tunneling shaft 32 may range from about 2 millimeters (mm) to about 15 mm, although other examples are contemplated. The length of tunneling shaft 32 from proximal end 36 directly adjacent handle 34 to distal end 38 may range from about 4 inches to about 12 inches, although other examples are contemplated. In examples in which a portion of tunneling shaft 32 is substantially straight from the proximal end 36 adjacent to handle and then transitions to a curved portion at a point between proximal end 36 and distal end 38, approximately ⅓ (one-third) of the length of tunneling shaft 32 out of proximal end 36 may be approximately straight. In some examples, tunneling shaft 32 may have a portion that is approximately straight for a length of about 0.5 inches to about 1.5 inches (e.g., in the case of tunneling shaft 32 having an overall length of about 4 inches). In some examples, tunneling shaft 32 may have a portion that is approximately straight for a length of about 3 inches to about 5 inches (e.g., in the case of tunneling shaft 32 having an overall length of about 12 inches). In some examples, approximately ⅔ (two-thirds) of the overall length of tunneling shaft 32 out of proximal end 36 may be approximately straight. In some examples, tunneling shaft 32 may have a portion that is approximately straight for a length of about 7 inches to about 9 inches (e.g., in the case of tunneling shaft 32 having an overall length of about 12 inches).

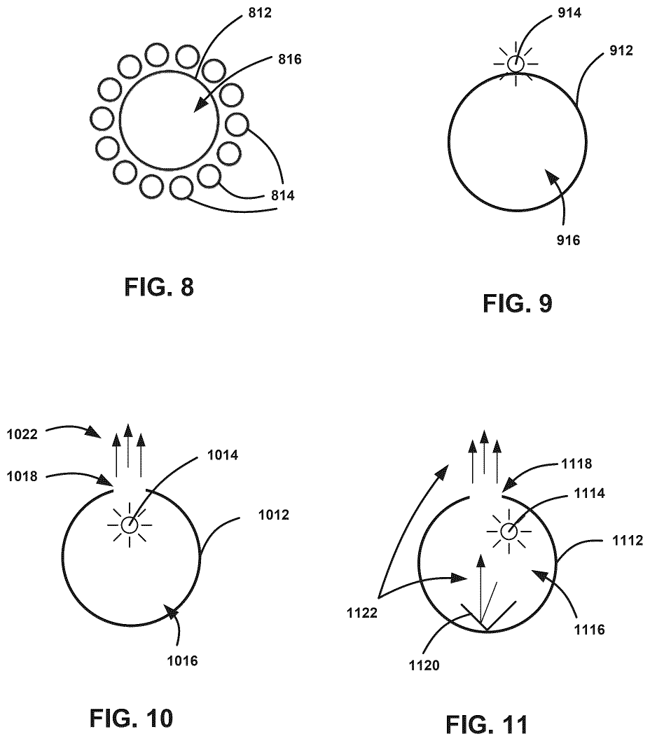

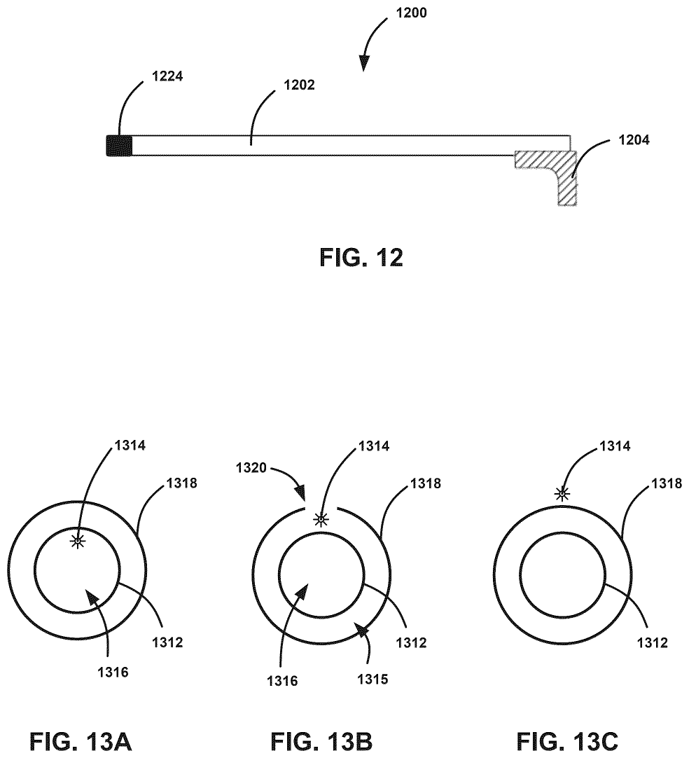

Tunneling shaft 32 defines an inner lumen 46 that extends from the proximal end 36 to distal end 38. One or more lighting elements (e.g., lighting element 52) may be disposed along the length of tunneling shaft 32 to allow direct visualization of tunneling shaft while inside the patient's body. While lighting element 52 according to the example of FIG. 3 is shown disposed on tunneling shaft 32, in some examples lighting element 52 may be disposed on a sheath that surrounds tunneling shaft 32 along the length of tunneling shaft 32, e.g., as described below with reference to FIGS. 12-13C. In the example of FIG. 3, distal end 38 has a dome shape for the leading edge to allow for blunt dissection. However, other shapes are contemplated. In some examples, during an implant procedure, an endoscope or other optical tool may be inserted into inner lumen 46 via proximal opening 48 in handle 34 and advanced through lumen 46 to distal end 38 of tunneling tool 32 adjacent optical window 44. Optical window 44 may be formed of a transparent material, for example glass, quartz or clear plastics like polycarbonate (e.g., LEXAN) or acrylic. In this manner, a surgeon or other user may visualize the path of distal end 38 when advanced through tissue of patient 12 during the insertion of tunnel tool 32 into patient 12. In some examples, a lighting element may be disposed within distal end 38 of tunneling tool 30 and, once activated, emit light through optical window 44. The light emitted by the lighting element (e.g., lighting element 52) may be sensed by an endoscope inserted in a pleural cavity of patient 12.

Tunneling tool 30 may include an activation element, such as a trigger 56 of handle 34 configured to activate the one or more lighting elements (e.g., lighting element 52). For example, tunneling tool 30 may include control circuitry such that actuation of trigger 56 causes the control circuitry to activate the one or more lighting elements. Although only one activation element is shown in FIG. 3, in some examples tunneling tool 30 may include multiple actuation elements. Trigger 56 may be selectively actuated by a user to turn on lighting element 52. Trigger 56 may also be configured to change an operation state of lighting element 52 in response to actuation. For example, trigger 56, when actuation, may cause control circuitry to change the operation state of lighting element 52. For example, lighting element 52 may be configured to emit light at different wavelengths, intensities, and or to blink (also “flash”), where repeated actuations of the activation element cause the control circuitry to cycle lighting element 52 through one or more states of emitting light at different wavelengths, intensities, and/or flashing intervals.

Handle 34 may be shaped such that when gripped by a hand of a surgeon or other operator, trigger 56 may be depressed or otherwise actuated by a finger such as the index finger in the case of tunneling tool 30. In other examples, handle 34 may be shaped such that when gripped by a hand of a surgeon or other operator, trigger 56 may be depressed or otherwise actuated by the thumb. In some examples, trigger 56 may be shielded by adjacent walls of handle 34 by recessing trigger 56 to some extent into the surface of handle, e.g., to protect against unwanted depression of trigger 56 during a tunneling procedure. In some examples, handle 34 may include a trigger guard to prevent trigger 56 from being depressed accidently by a surgeon or other operator during a tunneling procedure. In some examples, a vertical portion of handle 34 may be angled further towards distal end 38 of tunneling shaft 32. In some examples, the actuation element may be a button or other touch-responsive mechanism.

In some examples, the depression (pulling) of trigger 56 activates (e.g., causes control circuitry to activate) lighting element 52 from an OFF state to an ON state. In some examples, lighting element 52 may remain in the ON state until trigger 56 is released. In some examples, lighting element 52 may remain in the ON state until trigger 56 is depressed once more, or a number of times more. For example, repeated actuations of trigger 56 may cycle through a number of operation states before control circuitry turns off lighting element 52.

In some examples, tunneling tool 30 may include one or more cutting tools to assist the tunneling procedure as described in U.S. Patent Application Publication No. 2020/0038048 (Ebersole et al.), and U.S. Patent Application Publication No. 2022/0031390, both of which are incorporated herein by reference in their entirety.

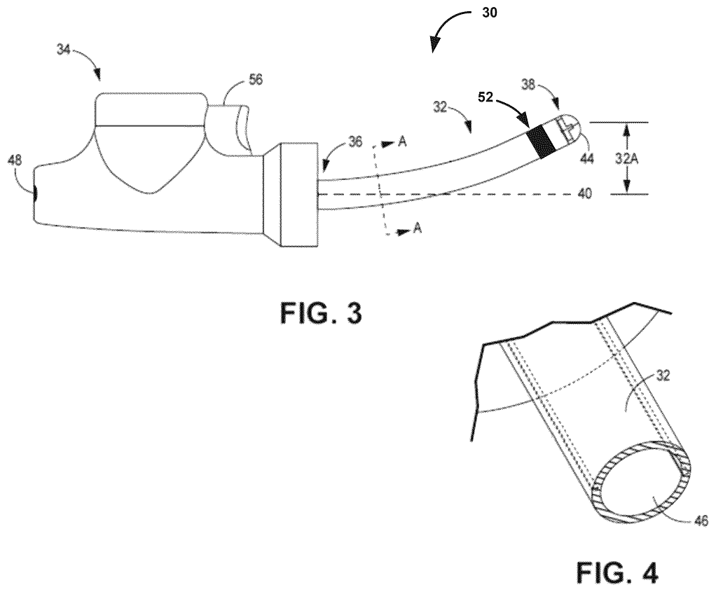

FIG. 5 is a conceptual diagram illustrating an example implant system 500 in accordance with the present disclosure. Implant system 500 may be used during an implant procedure on patient 12. Implant system 500 includes tunneling tool 510, and endoscope 520 in communication with computing device 526. Tunneling tool 510 may be substantially similar to tunneling tool 30 of FIG. 3. In some examples, a left lung of patient 12 is deflated during the implant procedure. Implant system may also include a lead (e.g., lead 16 of FIGS. 1A-1B) for implantation after a tunneling procedure is completed, and an implantable medical device (e.g., pulse generator 14 of FIG. 1A) configured to provide therapy via the lead.

As shown in FIG. 5, a shaft of endoscope 520 may be inserted into patient 12 through endoscopic incision 524, and be disposed within the left pleural cavity 35 of patient 12. A distal end of the endoscope shaft includes optical device 522 directed at a location of the anticipated tunneling trajectory for tunneling tool 510. Optical device 522 may be configured to sense light from lighting elements 524 during the implant procedure. In some examples, optical device 522 may be a camera configured to sense light at a variety of wavelengths and frequencies. Endoscope 520 may include processing circuitry (not shown) for receiving sensed signals from optical device 522 and sending signal data from the sensed signals to computing device 526. Computing device 526 may include processing circuitry configured to process the signal data into images viewable on a display of computing device 526 for an operator to visualize the interior of patient 12. When tunneling tool 510 is inserted into patient 12 and lighting elements 524 are activated, the operator may be able to visualize the placement and trajectory of tunneling tool 510 within patient 12, as well as internal body structures of patient 12. When describing the operator visualizing features of tunneling tool 516 inside patient 12, it may be understood that the operator is viewing the features on a display of computing device 526 based on sensed data from optical device 522 of endoscope 520.

Processing circuitry of computing device 526 may be configured to identify key tissues within patient 12 based on sensed data from optical device 522 of endoscope 520. For example, based on sensed data from optical device 522, computing device 526 may identify one or more of cardiac tissue, lung tissue, scar tissue, skeletal tissue, or other patient tissues. In some examples, computing device 526 may identify the one or more tissues of patient 12 based on silhouettes of the tissues due to illumination within patient 12 from lighting elements 524. In some examples, computing device 526 may identify the one or more tissues of patient 12 based on direct visualization of the tissues from optical device 522.

Processing circuitry of computing device 526 may be configured to determine a distance of the key tissues in patient 12 from one or more of tunneling tool 510 (e.g., tunneling shaft 514), and endoscope 520. Computing device 526 may determine if the distance satisfies a threshold. For example, if the distance is too small, computing device 526 may determine that the tunneling tool 510 or endoscope 520 are too close to the key patient tissues. In order to avoid damage to the key patient tissues, computing device 526 may be configured to cause one or more of lighting elements 524 to change an operation state in response to the distance satisfying a threshold. For example, computing device 526 may determine that the distance between a distal end of tunneling shaft 514 and heart 6 is at or below a threshold distance value. In response, computing device 526 may cause lighting element 524A to change from emitting light in a continuous state to emitting light in a flashing state. In some examples, computing device 526 may be configured to cause only the lighting element of lighting elements 524 closest to the calculated distance to change an operation state. In this way it may be easier to visualize the problem from a display of computing device 526. In some examples, computing device 526 may cause lighting elements 524 to change operation states in response to the determined distance satisfying a threshold in any of the ways described below with respect to actuation elements (e.g., light intensity, light wavelength, continuous state, flashing state). In this way, an operator of tunneling tool 510 may quickly recognize potential issues during an implant procedure.

In some examples, computing device 526 may be configured to output a visual or audio alert (e.g., on the display or speakers of computing device 526) in response to the distance satisfying the threshold. In some examples, the alert may depict and or describe the identified issue. For example, computing device 526 may output text on the display that reads “distal tip of tunneling shaft too close to heart.” In some examples, an audio alert may include words describing the identified issue, and or alarm tones.

In some examples, processing circuitry of computing device 526 may be configured to estimate a trajectory of tunneling shaft 514 and project the estimated trajectory onto the display. For example, tunneling shaft 514 may be at least partially inserted within patient 12, and computing device 526 may determine, based on an orientation of tunneling shaft 514 within patient 12, that continued advancement of tunneling shaft 514 will result in tunneling shaft 514 following a particular trajectory. Computing device 526 may output the estimated trajectory to a display using an augmented reality approach to allow a user to visualize potential paths of tunneling shaft 514 as it is advanced in patient 12. In some examples, computing device 526 may estimate the trajectory of tunneling shaft 514 based on an orientation of one or more of lighting elements 524. For example, lighting element 524A may allow computing device 526 to identify a distal tip of tunneling shaft 514. In some examples, lighting element 524B may be configured to emit light in a distinct radial direction away from tunneling shaft 514. Based on an intensity of light measured by optical device 522 corresponding to lighting element 524B, computing device 526 may determine an orientation of tunneling shaft 514 within patient 12.

A distal portion of tunneling shaft 514 is inserted through incision site 504, e.g., at access site A shown in FIG. 2, with the operator controlling the movement of shaft 514 by gripping handle 516, which is located externally. Handle 516 remains outside patient 12 to allow for a surgeon or other operator to maneuver tunneling shaft 514 along the desired path within the substernal space of patient 12. Distal end 518 of shaft 514 may be advanced superiorly, e.g., to the position shown in FIG. 5, to create a portion of a passageway and a sub-sternal tunnel. In some examples, the substernal space in which tunneling shaft 514 is inserted is a space within transverse thoracic muscles of patient 12. The surgeon or other operator may view the path of distal end 518 of tunneling shaft 514 during the procedure through optical device 522 of endoscope 520, with the assistance of lighting element 524A. In some examples, the operator may also view a path directly in front of distal end 518 by way of an additional viewing device inserted within an inner lumen of shaft 514. The surgeon or other operator may tunnel through tissue of patient 12 by way of blunt dissection using distal end 518 of tunneling shaft. In some examples, distal end 518 may also include one or more devices for sharp dissection as described above.

In some examples, tunneling shaft 514 may have a substantially oval (e.g., elongated oval) cross-section in a plane orthogonal to the longitudinal axis of tunneling tool 510. The oval cross-sectional shape may allow for substantially flat surfaces (“top” and “bottom” surfaces) of tunneling shaft 514 to be adjacent to the sternum inner surface and pericardial sac outer surface when utilized according to the tunneling techniques described herein. As described above with respect to FIG. 3, tunneling shaft 514 may be curved. The curvature of tunneling shaft 514 may bias the distal portion of tunneling shaft 514 towards an inner surface of a sternum of patient 12. In some example, the “flat upper” surface of shaft 514 may be guided or otherwise in contact with sternum inner surface during a tunneling procedure, which may provide for increased stability of tunneling tool 510 in the hand(s) of a surgeon or other operator.

One or more lighting elements (e.g., lighting elements 524A-C, together, lighting elements 524) may be disposed along the length of tunneling shaft 514. Although three lighting elements 524 are shown in the example of FIG. 5, tunneling tool 510 may include any one or more lighting elements. Lighting elements 524 may be disposed along a length of tunneling shaft 514 in locations corresponding to structure of tunneling tool 510, or structure of another device of implant system 500. For example, lighting element 524A may be disposed at a position along the length of tunneling shaft 514 corresponding to a distal end 518 of tunneling shaft 514. In some examples, a position of lighting element 524B may correspond to the location along the length of tunneling shaft 514 where an electrode of the lead of implant system 500 would be positioned after implantation of the lead. In some examples, a position of lighting element 524C may correspond to a location along the length of tunneling shaft 514 corresponding to an apex of a bend in tunneling shaft 514. In some examples, a position of lighting element 524A may correspond to both distal end 518 of tunneling shaft 514 and a location of a lead electrode within patient 12 after implantation of the lead. Due to the location of lighting elements 524 at key points along the length of tunneling shaft 514, an operator of implant system 500 may better visualize the location of tunneling shaft 514 inside patient 12 as sensed by optical device 522, even when patient tissues lie in between optical device 522 and tunneling shaft 514.

Lighting elements 524 may be configured to shine with a wavelength and/or intensity sufficient for optical device 522 to visualize lighting elements 524 through the patient's tissue. For example, lighting elements 524 may reveal not only the position of tunneling shaft 514 within patient 12, but also illuminate patient tissue structures (e.g., a cardiac silhouette, adhesions in tissue, arteries, and veins), bleeding spots, or other conditions inside the patient during the implant procedure. Visualization of these patient tissue structures may ensure an operator avoids undue adverse effects to patient 12 as a result of an implant procedure. In some examples, lighting element 524 may be configured to emit light with intensities ranging from 5 to 500 lumens. In some examples, control circuitry of tunneling tool 510 may be configured to cause lighting elements 524 to emit light in different intensities based on a wavelength of light emitted by lighting elements 524. For example, light in the visible spectrum may be easier to visualize through tissue if a wavelength of the light is on the shorter side of the visible spectrum (e.g., blue, indigo, violet) than light with a longer wavelength (e.g., red). Light emitted with higher intensity (e.g., higher lumens) may be easier to visualize through patient tissue than light emitted with less intensity. In some examples, control circuitry of tunneling tool 510 may be configured to cause lighting elements 524 that emit light with relatively longer wavelengths to emit light with relatively higher intensity, and cause lighting elements 524 that emit light with relatively shorter wavelengths to emit light with relatively lower intensity.

In some examples, different lighting elements 524 may be configured to emit light at a different wavelength or frequency. For example, the one or more lighting elements of implant system 50 may include a first lighting element (e.g., lighting element 524A) and a second lighting element (e.g., lighting element 524B), wherein lighting element 524A is configured to emit light in a green light spectrum and lighting element 524B is configured to emit light in a blue light spectrum. In some examples, each of the different lighting elements 524 may be configured to emit light at different corresponding wavelengths or frequencies. In some examples, processing circuitry of computing device 526 may be configured to amplify the differences between the colors of the emitted light for display on a display device of computing device 526. In this way, post-processing of the sensed light may make up for light scattering through patient tissue that could cause sensed light to appear a similar color. In some examples, lighting elements 524 may be configured to emit light with wavelengths in the visible spectrum shorter than the red spectrum. Emitted light with wavelengths on the shorter side of the visible spectrum may be more visible through tissue than emitted light with wavelengths on the longer side of the visible spectrum. In some examples, one or more of lighting elements 524 may be configured to emit light not in the visible spectrum. For example, lighting element 524C may be configured to emit near infrared light. Optical device 522 may be configured to sense light emitted by lighting elements 524 that is not in the visible spectrum. Computing device 526 may process signal data received from optical device 522 to allow an operator to see a representation of the emitted light on a display of computing device 526. The different wavelengths of light emitted by lighting elements 514 may help an operator differentiate between the locations of different features of tunneling tool 510 within patient 512. In some examples, each lighting element of lighting elements 524 may be configured to emit light at multiple different wavelengths or frequencies, and control circuitry 517 of tunneling tool 510 may be configured to cause lighting elements 524 to emit light at one or more of the different wavelengths or frequencies.

In some examples, different lighting elements 524 may be configured to emit light at a different intensity. For example, a first lighting element 524A may be configured to emit light at a higher intensity (brighter) than a second lighting element 524B. In some examples, each of lighting elements 524 may be configured to emit light at a variety of intensities. For example, at a first time, control circuitry 517 may control lighting element 524A to emit light at a high intensity, and lighting element 524B to emit light at a low intensity. At a second time, control circuitry 517 may control lighting element 524A to emit light at a low intensity, and lighting element 524B to emit light at a high intensity.

In some examples, one or more of lighting elements 524 may be configured to emit light continuously. In some examples, one or more of lighting elements 524 may be configured to emit light in flashes over predetermined time intervals. For example, lighting element 524A may be configured to blink every second and lighting element 524B may be configured to emit a continuous stream of light. In some examples, each of lighting elements 524 may be configured to both emit light continuously and emit light in flashes over predetermined time intervals. Control circuitry 517 may control each of lighting elements 524 to emit light either continuously or in flashes at different times. For example, at a first time, control circuitry 517 may control lighting element 524A to emit light continuously, and control lighting element 524B to emit light in flashes at 0.5 second time intervals. At a second time, control circuitry 517 may control lighting element 524A to emit light in flashes at 0.2 second time intervals, and control lighting element 524B to emit light continuously.

In some examples, control circuitry 517 may be configured to cause different lighting elements 524 to flash at different time intervals. In some examples, control circuitry 517 may cause lighting elements corresponding to locations along the length of tunneling shaft 514 where electrodes of a lead will be implanted to flash at a first predetermined time interval. In some examples, control circuitry 517 may cause lighting elements that do not correspond to electrode locations to flash at a second predetermined time interval different than the first predetermined time interval. In some examples, control circuitry 517 may cause each of the different lighting elements 524 to flash at different time intervals. A flash of light may include certain characteristics. For example, characteristics of a flash of light may include a first period of time in which the light is emitted by a particular lighting element and a second period of time in which the light is not emitted by the particular lighting element during a single flashing event. In some examples the first period of time and the second period of time are the same duration. In some examples, the first period of time and the second period of time are different durations. In some examples, characteristics of a flash of light may include an intensity profile of the emitted light over a period of time in which light is emitted by a particular lighting element (e.g., the first period of time). For example, when the particular lighting element begins emitting light again during the first period of time, it may not emit light at a consistent intensity. The intensity of the light may gradually increase and then gradually decrease during the first period of time. In some examples, the increase duration may be equal to or different from the decrease duration. The different flashing intervals and flashing characteristics for light emitted by different lighting elements 514 may help an operator differentiate between the locations of different features of tunneling tool 510 within patient 512.

In some examples, control circuitry 517 may be configured to cause lighting elements 524 to emit light in different operation states. For example, control circuitry 517 may cause lighting elements 524 to emit light in a continuous state, in which lighting elements 524 emit a continuous stream of light. In some examples, control circuitry 517 may cause lighting elements 524 to emit light in a flashing state, in which lighting elements 524 emit flashes of light at different time intervals. In some examples, control circuitry 517 may cause lighting elements 524 to emit light in a fast flashing state, a medium flashing state, and/or a slow flashing state. A lighting element in the fast flashing state may emit light in flashes at shorter intervals than a lighting element in the medium flashing state, and a lighting element in the medium flashing state may emit light in flashes at shorter intervals than a lighting element in the slow flashing state. In some examples, control circuitry 517 may cause lighting elements 524 to emit light in a first color state, and a second color state, wherein the wavelength of light emitted is different for each color state.

In some examples, lighting elements 524 may be configured to emit light in a particular wavelength. For example, lighting element 524A may be a red LED, lighting element 524B may be a blue LED, and lighting element 524C may be a green LED. In some examples, lighting elements 524 may be configured to emit light in any one of multiple wavelengths. Control circuitry 517 may be configured to cause lighting elements 524 to emit light in one or more of the multiple wavelengths. For example, at a first time, control circuitry 517 may cause lighting element 524A to emit a red light, lighting element 524B to emit a blue light, and lighting element 524C to emit a green light. At a second time, control circuitry 517 may cause lighting element 524A to emit a yellow light, lighting element 524B to emit a purple light, and lighting element 524C to emit a red light.

Control circuitry 517 may be configured to change an operation state of lighting elements 514 in response to one or more stimuli. For example, handle 516 of tunneling tool 510 may include actuation element 515 that, when actuated, causes control circuitry 517 to activate lighting elements 524. When activated, lighting elements 524 may be in an ON-state and emitting light according to one or more operation states. In some examples, control circuitry 517 may cause lighting elements 514 to change operation states in response to actuation of actuation element 515. For example, a first actuation of actuation element 515 may cause control circuitry 517 to activate one or more of lighting elements 514, and successive actuations of actuation element 515 may cause control circuitry 517 to change the operation state of one or more of lighting elements 514. For example, a first actuation of actuation element 515 may, by way of control circuitry 517, cause lighting element 524A to emit light in a continuous state, and a second actuation of actuation element 515 may, by way of control circuitry 517, cause lighting element 524A emit light in a flashing state. Description of actuation element 515 causing an effect to one or more of lighting elements 514 may be understood to include control circuitry 517 causing the effect to lighting elements 514 in response to actuation of actuation element 515.

In some examples a first actuation of actuation element 515 may cause lighting element 524A to emit light in a continuous state or a flashing state, while lighting element 524B remains in an OFF-state, and a second actuation of actuation element 515 may cause lighting element 524B to begin emitting light in either a continuous state or a flashing state. In some examples a first actuation of actuation element 515 may cause lighting element 524A to emit light in a continuous state, a second actuation of actuation element 515 may cause lighting element 524A to emit light in a slow flashing state, and a third actuation of actuation element 515 may cause lighting element 524A to emit light in a fast flashing state. In some examples a first actuation of actuation element 515 may cause lighting element 524A to emit light in a first color state, a second actuation of actuation element 515 may cause lighting element 524A to emit light in a second color state, where the wavelength of light is different between the first color state and second color state. In some examples, one or more of lighting elements 524 may be in a combination of a color state and either a continuous state or a flashing state. For example, in a first actuation of actuation element 515 may cause lighting element 524A to emit light in a continuous state and a first color, and a second actuation of actuation element 515 may cause lighting element 524A to emit light in a slow flashing state and a second color. In some examples, lighting elements 524 may go through a cycle of operation states before returning to an OFF-state. In some examples, lighting elements may only have one operation state during an ON-state before returning to an OFF-state. Any combination of lighting elements 524 and operation states is contemplated in any order of actuations of actuation element 515.

Although described above with respect to a single actuation element 515, in some examples tunneling tool 510 includes a plurality of actuation elements, wherein each actuation element controls either a different lighting element of lighting elements 524 or a different operation state of one or more lighting elements of lighting elements 524. For example, each of lighting elements 524 may have a dedicated actuation element, the actuation of which causes control circuitry 517 to adjust an operation state of the corresponding lighting element. In some examples, different actuation elements may cause control circuitry 517 to adjust all lighting elements 524 to particular operation states. For example, tunneling tool 510 may include an activation actuation element that, when actuated, causes control circuitry 517 to activate one or more of lighting elements 524. In some examples, tunneling tool 510 may include a separate, flashing-state actuation element that, when actuated, causes control circuitry 517 to change the operation state of one or more of lighting elements 524 to a flashing state. In some examples, tunneling tool 510 may include a separate color-state actuation element that, when actuated, causes control circuitry 517 to change the color state of one or more of lighting elements 524. Any combination of actuation elements to lighting elements and operation states is contemplated.

Control circuitry 517 may be formed in one or more microprocessors, application specific integrated circuits (ASICs), field programmable gate arrays (FPGAs), digital signal processors (DSPs), fixed function circuitry, programmable processing circuitry, various combinations of fixed function circuitry with programmable processing circuitry, or other equivalent integrated logic circuitry or discrete logic circuitry. Fixed-function circuitry refers to circuits that provide particular functionality and are preset on the operations that can be performed. Programmable processing circuitry refers to circuits that can be programmed to perform various tasks and provide flexible functionality in the operations that can be performed. For instance, programmable processing circuitry may represent hardware that executes software or firmware that cause programmable circuits to operate in the manner defined by instructions of the software or firmware. Fixed-function circuitry may execute software instructions (e.g., to receive parameters or output parameters), but the types of operations that the fixed-function processing circuits perform are generally immutable. In some examples, one or more of the units may be distinct circuit blocks (fixed-function or programmable), and in some examples, the one or more units may be integrated circuits. In some examples, tunneling tool 510 may include a power source for powering control circuitry 517 and lighting elements 524.

In some examples, control circuitry 517 for operating lighting elements 524 may be disposed within handle 516 of tunneling tool 510. In some examples, one or more of control circuitry 517 or actuation elements for lighting elements 524 may be housed in a separate device and operated independently of tunneling tool 510. For example, while the lighting elements 524 have been described as a part of tunneling tool 510, in some examples implant system 500 may include a lighting control device separate from tunneling tool 510. The lighting control device may include control circuitry for lighting elements 524 and one or more actuation elements for lighting elements 524. The lighting control device may also include a separate shaft that incorporates lighting elements 524 that is inserted along with tunneling tool 510 during an implant procedure. In some examples, the lighting control device may be a remote. The one or more activation elements may be positioned on the remote for actuation by one or more of a user's fingers when the user grips the remote.

In some examples, tunneling tool 510 may include a pressure sensor to sense a pressure inside patient 12. In some examples, the pressure sensor may be configured to measure a force pressure with which part of the tunneling tool is pressing against patient tissue. For example, tunneling tool 510 may include a pin and spring at distal end 518 or another location along shaft 514, where a sufficient force acting on the pin from the patient's tissue overcomes the force of the spring and depresses the pin. In some examples, the pressure sensor may be configured to measure a blood pressure of patient 12. Control circuitry 517 may be configured to change an operation state of lighting elements 524 in response to a sensed pressure satisfying a threshold. For example, in response to sensing that a force pressure at the distal end 518 exceeds a threshold pressure, control circuitry 517 may cause lighting element 524A to emit light in a fast flashing state and/or a red color state. In some examples, in response to sensing that a blood pressure of patient 12 exceeds a threshold blood pressure, control circuitry 517 may cause each of lighting elements 524 to emit light in different flashing states and in different color states. In some examples, control circuitry 517 may cause lighting elements 524 to change operation states in response to pressure in any of the ways described above with respect to actuation elements. In this way, an operator of tunneling tool 510 may quickly recognize potentially adverse conditions during an implant procedure.

Although endoscope 520 is shown in FIG. 5 in a left pleural cavity of patient 12, in some examples endoscope 520 may be inserted in patient 12 in other positions. In some examples, endoscope 520 may be inserted into a substernal space of patient 12. In some examples, the substernal space in which endoscope 520 is inserted is a space within transverse thoracic muscles of the patient. In some examples, endoscope 520 and tunneling shaft 514 may be advanced in a parallel fashion. For example, both endoscope 520 and tunneling shaft 514 of tunneling tool 510 may be inserted sub-xiphoid and advanced parallel to one another within patient 12. For example, tunneling tool 510 may be directly visible by optical device 522 of endoscope 520. In some examples, both endoscope 520 and tunneling shaft 514 may be advanced together. In some examples, endoscope 520 may be advanced separately from tunneling shaft 514.

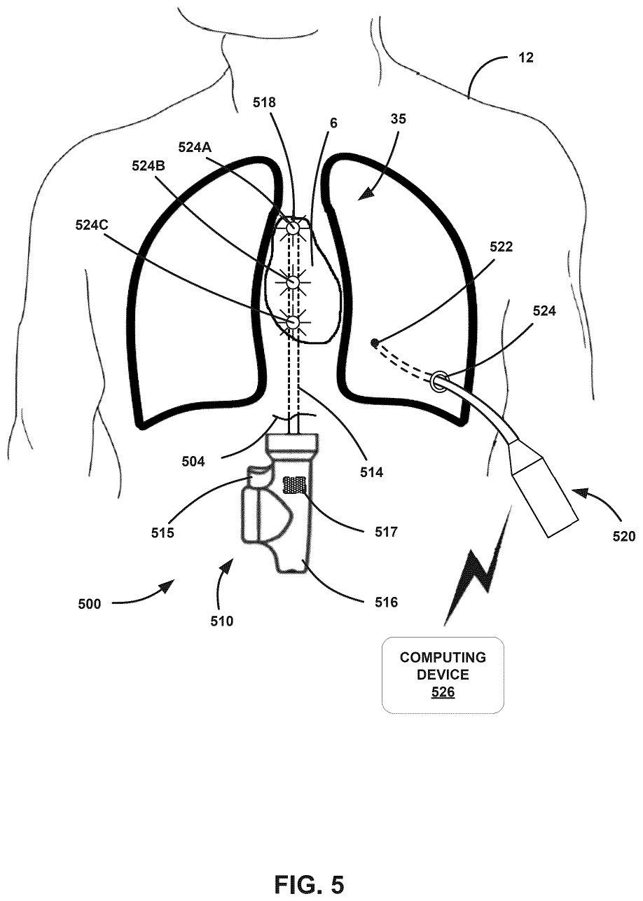

FIG. 6 is a conceptual diagram illustrating example computing device 526 of system 500 of FIG. 5. Computing device 526 includes processing circuitry 602, memory 604, display 606, and power source 608. Computing device 526 may be in communication with one or more of endoscope 520, tunneling tool 510, and/or another computing device.

Processing circuitry 602 may be formed in one or more microprocessors, application specific integrated circuits (ASICs), field programmable gate arrays (FPGAs), digital signal processors (DSPs), fixed function circuitry, programmable processing circuitry, various combinations of fixed function circuitry with programmable processing circuitry, or other equivalent integrated logic circuitry or discrete logic circuitry. Fixed-function circuitry refers to circuits that provide particular functionality and are preset on the operations that can be performed. Programmable processing circuitry refers to circuits that can be programmed to perform various tasks and provide flexible functionality in the operations that can be performed. For instance, programmable processing circuitry may represent hardware that executes software or firmware that cause programmable circuits to operate in the manner defined by instructions of the software or firmware. Fixed-function circuitry may execute software instructions (e.g., to receive parameters or output parameters), but the types of operations that the fixed-function processing circuits perform are generally immutable. In some examples, one or more of the units may be distinct circuit blocks (fixed-function or programmable), and in some examples, the one or more units may be integrated circuits. As shown in FIG. 6, processing circuitry 602 is communicatively coupled to memory 604 and display 606.

Memory 604 may store instructions for execution of one or more applications. Memory 604 may include one or more computer-readable storage media (e.g., a non-transitory computer-readable storage medium), computer-readable storage devices, etc. Examples of memory devices include, but are not limited to, a random access memory (RAM), an electrically erasable programmable read-only memory (EEPROM), flash memory, or other medium that can be used to carry or store desired program code in the form of instructions and/or data structures and that can be accessed by a computer or one or more processors (e.g., processing circuitry 602).

In some examples, memory 604 may store instructions that cause processing circuitry 603 to perform the functions ascribed in this disclosure to the processing circuitry. Accordingly, at least one device of memory devices comprising memory 604 may represent a computer-readable storage medium having instructions stored thereon that, when executed, cause one or more processors (e.g., the processing circuitry) to perform various functions. For instance, at least one of the memory devices is a non-transitory storage medium. The term “non-transitory” indicates that the storage medium is not embodied in a carrier wave or a propagated signal. However, the term “non-transitory” should not be interpreted to mean that the memory devices are non-movable or that the stored contents are static. As one example, at least one of the memory devices described herein can be removed from computing device 526, and moved to another device.

Processing circuitry 602 may be configured to communicate with one or more computing devices using network interface hardware. Examples of network interface include a direct interface or a transitive interface to a network, such as a wireless or wired network. In cases of a direct interface to a wireless network, such interface hardware may include, be, or be part of various wireless communication hardware, including, but not limited to, one or more of Bluetooth®, 3G, 4G, 5G, or WiFi® radios. In cases of a wired network or a first link in a transitive interface to a wireless network, the interfaces may incorporate wired communication hardware, wireless communication hardware (or some combination thereof), such as any one or any combination of a network interface card (e.g., an Ethernet card and/or a WiFi® dongle), USB hardware, an optical transceiver, a radio frequency transceiver, Bluetooth®, 3G, 4G, 5G, or WiFi® radios, and so on.

Processing circuitry 602 may receive signal data from endoscope 520 indicative of a visualization inside patient 12. For example, signal data may include sensed signals from a camera of endoscope 520 inside patient 12. Processing circuitry 602 may be configured to process the signal data into images viewable on display 606 for an operator to visualize the interior of patient 12. In some examples, as shown in FIG. 5, a camera of endoscope 520 may be configured to sense signals corresponding to one or more lighting elements of a tunneling tool. The one or more lighting elements may be configured to emit light at different wavelengths. Processing circuitry 602 of computing device 526 may be configured to amplify the differences between the colors of the emitted light from the one or more lighting elements when outputting an image to display 606. In some examples, processing circuitry 602 may be configured to receive sensed data indicative of light outside of the visible spectrum, and output an image to display 606 that includes a representation of that light in the visible spectrum. In some examples, processing circuitry 602 may be configured to project an image of the complete tunneling tool on to display 606 based on the location of two or more light sources.

Processing circuitry 602 may be configured to identify key tissues within patient 12 based on sensed data from an optical device of an endoscope. For example, based on sensed data from the optical device, processing circuitry 602 may identify one or more of cardiac tissue, lung tissue, scar tissue, skeletal tissue, or other patient tissues. In some examples, processing circuitry 602 may identify the one or more tissues of the patient based on silhouettes of the tissues due to illumination within the patient from one or more lighting elements along the length of a tunneling shaft of a tunneling tool. In some examples, processing circuitry 602 may identify the one or more tissues of the patient based on direct visualization of the tissues from the optical device.

Processing circuitry 602 may be configured to determine a distance of the key tissues in patient 12 from one or more of a tunneling tool and/or an endoscope. Processing circuitry 602 may determine if the distance satisfies a threshold. For example, if the distance is too small, processing circuitry 602 may determine that the tunneling tool or endoscope are too close to the key patient tissues. In order to avoid damage to the key patient tissues, processing circuitry 602 may be configured to cause one or more of the lighting elements to change an operation state in response to the distance satisfying a threshold. For example, processing circuitry 602 may determine that the distance between a distal end of the tunneling shaft and a heart is at or below a threshold distance value. In response, processing circuitry 602 may cause a lighting element on a distal end of the tunneling shaft to change from emitting light in a continuous state to emitting light in a flashing state. In some examples, processing circuitry 602 may be configured to cause only the lighting element closest to the calculated distance to change an operation state. For example, as in the previous example, the distal end of the tunneling shaft was determined to be too close to the heart, therefore processing circuitry 602 caused the lighting element on the distal end of the tunneling shaft to change an operation state. In this way it may be easier to visualize the problem from a display of computing device 526. In some examples, computing device 526 may cause lighting elements to change operation states in response to the determined distance satisfying a threshold in any of the ways described above with respect to actuation elements (e.g., light intensity, light wavelength, continuous state, flashing state). In this way, an operator of a tunneling tool may quickly recognize potential issues during an implant procedure.

In some examples, processing circuitry 602 may be configured to output a visual or audio alert (e.g., on display 606 or speakers of computing device 526) in response to the distance satisfying the threshold. In some examples, the alert may depict and or describe the identified issue. For example, processing circuitry 602 may output text on display 606 that reads “distal tip of tunneling shaft too close to heart.” In some examples, an audio alert may include words describing the identified issue, and or alarm tones.

In some examples, processing circuitry 602 may be configured to estimate a trajectory of a tunneling shaft and project the estimated trajectory onto display 606. For example, the tunneling shaft may be at least partially inserted within patient 12, and processing circuitry 602 may determine, based on an orientation of the tunneling shaft within patient 12, that continued advancement of the tunneling shaft will result in the tunneling shaft following a particular trajectory. Processing circuitry 602 may output the estimated trajectory to display 606 using an augmented reality approach to allow a user to visualize potential paths of the tunneling shaft as it is advanced in patient 12. In some examples, processing circuitry 602 may estimate the trajectory of the tunneling shaft based on an orientation of one or more of the lighting elements. For example, lighting element 524A may allow processing circuitry 602 to identify a distal tip of tunneling shaft 514. In some examples, lighting element 524B may be configured to emit light in a distinct radial direction away from tunneling shaft 514. Based on an intensity of light measured by optical device 522 corresponding to lighting element 524B, processing circuitry 602 may determine an orientation of tunneling shaft 514 within patient 12.

In some examples, computing device 526 may be in communication with lighting circuitry of the one or more lighting elements. Computing device 526 may include one or more actuation devices for controlling an activation (ON-OFF) of the one or more lighting elements, as well as the operating state (intensity, wavelength, continuous state, flashing state) of the one or more lighting elements. For example, processing circuitry 602 may activate, deactivate, or change an operating state of one or more of the one or more lighting elements in response to actuation of one or more of the one or more actuation devices.

FIGS. 7A-&B are conceptual diagrams illustrating example tunneling devices 710 and 711 including example lighting elements 714A-B (together, lighting elements 714), and lighting element 715 in accordance with the present disclosure. Tunneling device 710, handle 716, button 720, guide member 718 and tunneling shaft 712 may be substantially similar, respectively, to tunneling device 711, handle 717, button 721, guide member 719 and tunneling shaft 713.

Tunneling tool 710 may include guide member 718 extending from handle 716 adjacent to and coplanar with tunneling shaft 712. Tunneling shaft 712 may be curved towards guide member 718. Handle 716 has a shape configured to receive fingers of a hand of a surgeon or other operator but may have any other suitable configuration for gripping. When gripped by the surgeon or other operator, a finger such as the index finger may be located in a manner that allows the finger to easily depress and release an actuation element (e.g., button 720) of tunneling tool 710.

During a procedure to gain sub-sternal access and create a sub-sternal tunnel in a patient, guide member 718 may help a surgeon or other operator in advancing tunneling shaft 712, once a distal end of tunneling shaft 712 is inserted into a patient. In some examples, curved distal portion 724 of guide member 718 may be configured to ‘ride’ on the skin over the sternum of the patient without binding on the skin during such a procedure. For example, curved distal portion 724 may be curved away from a sternum of a patient during use of tunneling tool 710. In this manner, guide member 718 may limit the depth below the sternum that tunneling shaft 712 may be advanced during the tunneling procedure. Further, the curvature of tunneling shaft 712 toward guide member 718 can cause the distal end of tunneling shaft 712 to ‘ride’ adjacent an inside surface of the sternum during the superior advancement thereof as an additional aid to the operator. In some example, the distance 726 between guide member 718 and tunneling shaft 712 may be adjusted as desired by a surgeon or other operator, e.g., based on the physical characteristics of a patient. For example, a proximal portion of guide member 718 attached to handle 716 may be configured to rotate. In some examples, tunneling shaft 712 may be sufficiently malleable to allow an operator to bend tunneling shaft 712. Tunneling shaft 712 may be sufficiently rigid to hold its shape after being bent by an operator. Examples of guide members 718 employed in a tunneling tool may include those described in U.S. patent application Ser. No. 15/204,579, by Malewicz et al., the entire content of which is incorporated herein by reference.

Lighting elements 714 may be disposed along the length, L, of tunneling shaft 712. Although four lighting elements 714 are shown in the example of FIG. 7A, tunneling tool 710 may include any one or more lighting elements. Lighting elements 714 may be disposed along length, L, of tunneling shaft 712 in locations corresponding to structures of tunneling tool 710, or structures of another device of an implant system. For example, lighting element 714A may be disposed at a position along the length of tunneling shaft 712 corresponding to a distal end of tunneling shaft 712. In some examples, a position of lighting element 714B may correspond to the location along the length of tunneling shaft 712 where an electrode of a lead will be disposed after implantation of said lead. In some examples, a position of lighting element 714C may correspond to a location along the length, L, of tunneling shaft 712 corresponding to an apex of a bend 722 in tunneling shaft 712. In some examples, bend 722 may separate a distal end of tunneling shaft 712 from a substantially straight proximal portion of tunneling shaft 712. In some examples, a position of lighting element 714D may correspond to a distal portion of the substantially straight portion of tunneling shaft 712. Due to the location of lighting elements 714 at key points along the length, L, of tunneling shaft 712, an operator of tunneling tool 710 may better visualize the location of tunneling shaft 712 inside a patient as sensed by a separate optical device, even when patient tissues lie in between the optical device and tunneling shaft 712.

Although bend 722 is shown in FIGS. 7A-B as a sudden curve in tunneling shaft 712, in some examples, bend 722 may form a curve about the entire length, L, from a proximal end of tunneling shaft 712 to a distal end (e.g., as shown in FIG. 3). In some examples, bend 722 may form a curve along a limited length of tunneling shaft 712 less than the entire length, L. In these examples: lighting element 714D may correspond to a location along length, L, where a proximal straight portion of tunneling shaft 712 transitions to a curved portion of tunneling shaft 712; lighting element 714C may correspond to an apex of the curved portion of tunneling shaft 712; and lighting element 714B may correspond to a distal end of the curved portion of tunneling shaft 712 and/or the beginning of a distal straight portion of tunneling shaft 712.

In some examples, lighting elements 714 may be separate from tunneling shaft 712 and inserted through an interior lumen of tunneling shaft 712 to their respective locations. For example, lighting elements 714 may be disposed on a separate shaft that is inserted into tunneling shaft 712. Handle 716 may include an opening connected to a proximal end of tunneling shaft 712 located inside handle 716 through which lighting elements 714 may be inserted. In some examples, an endoscope or other tool containing lighting elements 714 may be inserted into the opening of handle 716 and advanced, e.g., through the inner lumen of tunneling shaft 712 to or near a distal end of tunneling shaft 712. Tunneling shaft 712 may also include additional lumens, which may be used, e.g., for insufflation, irrigation, adding contrast, flushing lens, removing air pockets, and the like.

In examples where lighting elements 714 are disposed inside the lumen of tunneling shaft 712, tunneling shaft 712 may be configured to allow light from lighting elements 714 inside the lumen of tunneling shaft 712 to shine outside of tunneling shaft 712. For example, portions of tunneling shaft 712 may be made of clear or otherwise transparent material so as to allow light from lighting elements 714 to emit through the transparent section of tunneling shaft 712. In some examples, tunneling shaft 712 may be comprised primarily of one or more metals (e.g., stainless steel, coated steel, titanium alloys, aluminum alloys and others), with one or more windows in the metal material of tunneling shaft 712 through which light may be emitted. In some examples, the one or more windows may be wholly or partially covered with a transparent material (e.g., epoxy).

Tunneling tool 30 may include an actuation element, such as a button 720 of handle 716 configured to activate one or more of lighting elements 714. For example, actuation of button 720 may cause control circuitry of tunneling tool 710 to activate one or more of lighting elements 714. Although only one actuation element is shown in FIGS. 7A-B, in some examples tunneling tool 710 may include multiple actuation elements. Button 720 may be selectively actuated by a user to turn on lighting elements 714. Button 720 may also be configured to cause control circuitry of tunneling tool 710 to change an operation state of lighting elements 714 in response to actuation of button 720. For example, control circuitry of tunneling tool 710 may be configured to cause lighting elements 714 to emit light at different wavelengths, intensities, and or to blink (also “flash”) in response to actuation of button 720. In some examples, repeated actuations of the actuation element may cause control circuitry to cycle one or more of lighting elements 714 through one or more states of emitting light at different wavelengths, intensities, and/or flashing intervals.

In some examples, lighting elements 714 may include discrete light components. For example, lighting elements 714 may include discrete LEDs. In some examples, lighting elements 714 may include a fiberoptic component. In some examples, lighting elements 714 may include a laser.

In some examples, as shown in FIG. 7B, the one or more lighting elements of tunneling tool 711 may include a continuous linear light source along at least a portion of the length of tunneling shaft 713. Lighting element 715 is shown as a continuous linear light source along a distal portion of tunneling shaft 713. Proximal portion 705 of tunneling shaft 713 may not include any light sources. The position of lighting element 715 may correspond to key structures of tunneling shaft 713. For example, lighting element 715 may traverse a length of tunneling shaft 713 that includes a distal end of tunneling shaft 713 and a bend in tunneling shaft 713. Lighting element 715 may be configured to emit light at different wavelengths, intensities, and or flashing intervals along a length of lighting element 715. Lighting element 715 may be configured to emit light in different operating states in response to actuation of button 721.

In some examples, tunneling tool 711 may include pressure sensor 704 to sense a pressure inside a patient. In some examples, pressure sensor 704 may be configured to measure a force pressure with which part of tunneling tool 711 is pressing against patient tissue. For example, tunneling tool 711 may include a pin and spring at a distal end of tunneling shaft 713 or another location along shaft 713, where a sufficient force acting on the pin from the patient's tissue overcomes the force of the spring and depresses the pin. In some examples, pressure sensor 704 may be configured to measure a blood pressure of the patient. Control circuitry of tunneling tool 711 may be configured to change an operation state of lighting elements 715 in response to a sensed pressure satisfying a threshold. In some examples, the control circuitry may be configured to change the operation state of lighting elements 715 in response to a sudden change in sensed pressure. Although shown as part of tunneling tool 711 of FIG. 7B, in some examples, pressure sensor 704 may be included in any example arrangement of tunneling tools in accordance with the present disclosure.

In some examples, tunneling tool 711 may include impedance sensor 705 to sense an impedance inside a patient. In some examples, impedance sensor 704 may be configured to measure an impedance of tissue against which tunneling tool 711 is pressing. Control circuitry of tunneling tool 711 may be configured to change an operation state of lighting elements 715 in response to a sensed impedance satisfying a threshold. In some examples, the control circuitry may be configured to change the operation state of lighting elements 715 in response to a sudden change in impedance. Although shown as part of tunneling tool 711 of FIG. 7B, in some examples, impedance sensor 705 may be included in any example arrangement of tunneling tools in accordance with the present disclosure.

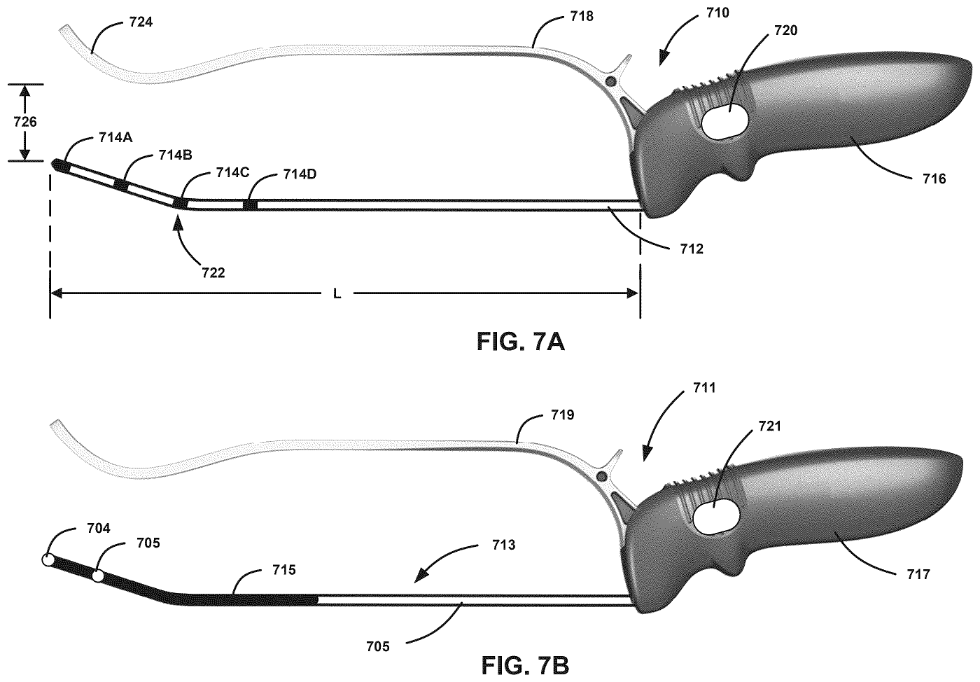

FIGS. 8-11 are conceptual diagrams illustrating example lighting elements with respect to cross sections of example tunneling shafts in accordance with the present disclosure. In some examples, one or more lighting elements may be disposed on an exterior of a tunneling shaft, as shown in FIGS. 8-9. In some examples, one or more lighting elements may be disposed on an interior of a tunneling shaft, as shown in FIGS. 10-11. In some examples, as shown in FIG. 8, one or more lighting elements may be configured to emit light in all radial directions away from a tunneling shaft. In some examples, as shown in FIGS. 9-11, one or more lighting elements may be configured to emit light in a distinct, radial direction away from a tunneling shaft.

In the example of FIG. 8, lighting element 814 is disposed on tunneling shaft 812 outside a shaft lumen 816 defined by tunneling shaft 812. In some examples, lighting element 814 may include a fiberoptic component. For example, in the example of FIG. 8, lighting element 814 is a fiber optic array encircling tunneling shaft 812. In some examples, lighting element 814 may be incorporated into tunneling shaft 812. For example, lighting element 814 may be attached to an exterior of tunneling shaft 812 by epoxy or another appropriate method. The epoxy may also serve to insulate lighting element 814 and/or wiring thereof from body fluid and/or tissue. In some examples, one or more grooves may be machined into an exterior of tunneling shaft 812 to house lighting element 814 or wiring thereof. In some examples, (e.g., in examples where lighting elements are disposed outside of the tunneling shaft) tunneling shaft 812 may include one or more ports that allow wiring of lighting element 814 to enter shaft lumen 816 at or near the location along tunneling shaft 812 where lighting element 814 is disposed. The wiring of lighting element 814 may travel through shaft lumen 816 proximal to a handle of the tunneling tool that includes tunneling shaft 812. Lighting element 814 may be configured to emit light in all directions radially away from tunneling shaft 812.

In the example of FIG. 9, lighting element 914 is disposed on tunneling shaft 912 outside a shaft lumen 916 defined by tunneling shaft 912. In the example of FIG. 9, lighting element 914 is an LED attached to an exterior of tunneling shaft 912. Lighting element 914 may be configured to emit light only on one half of tunneling shaft 912. For example, the LED of lighting element 914 may be attached to one side of tunneling shaft 912, and tunneling shaft 912 may be opaque. In this way, tunneling shaft 912 may substantially block light from lighting element 914 in a radial direction opposite the location where lighting element 914 is attached to tunneling shaft 912. As seen by an optical device separate from tunneling shaft 912, the intensity of light measured by the optical device may increase as tunneling shaft 912 is rotated until the optical device is located directly radially away from tunneling shaft 912 in the radial direction where lighting element 914 is disposed on tunneling shaft 912.

In the example of FIG. 10, lighting element 1014 is disposed inside shaft lumen 1016 of tunneling shaft 1012. Lighting element 1014 may be configured to emit light in a distinct, radial direction away from tunneling shaft 1012. For example, tunneling shaft 1012 may be comprised primarily of an opaque material, e.g., metal. A window 1018 may allow light 1022 emitted by lighting element 1014 to exit shaft lumen 1016 in a distinct radial direction away from tunneling shaft 1012. Light emitted in other directions may be blocked or suppressed by the opaque material of tunneling shaft 1012. In some examples, window 1018 may be covered by one or more translucent or transparent materials to seal shaft lumen 1016 from body fluid (e.g., epoxy), while allowing light 1022 to exit shaft lumen 1016 in the distinct radial direction.

In the example of FIG. 11, lighting element 1114 is disposed inside shaft lumen 1116 of tunneling shaft 1112. Lighting element 1114 may be configured to emit light in a distinct, radial direction away from tunneling shaft 1112. For example, tunneling shaft 1112 may be comprised primarily of an opaque material, e.g., metal. A window 1118 may allow light 1122 emitted by lighting element 1114 to exit shaft lumen 1116 in a distinct radial direction away from tunneling shaft 1112. Light emitted in other directions may be blocked or suppressed by the opaque material of tunneling shaft 1012. In some examples, window 1118 may be covered by one or more translucent or transparent materials to seal shaft lumen 1116 from body fluid (e.g., epoxy), while allowing light 1122 to exit shaft lumen 1116. In the example of FIG. 11, one or more reflective panels 1120 may be disposed inside shaft lumen 1116 configured to focus light emitted by lighting element 1114 out window 1118 in the distinct radial direction. The one or more reflective panels may be disposed on part of, or an entirety of an interior surface of tunneling shaft 1112 in the longitudinal position along tunneling shaft 1112 where window 1118 is disposed. In some examples, the one or more reflective panels 1120 may be attached to lighting element 1114. Although reflective panels 1120 are shown in FIG. 11 as disposed within shaft lumen 1116, in some examples, one or more lighting elements and one or more reflective panels may be disposed on an exterior of a tunneling shaft.

FIG. 12 is a conceptual diagram of an example introducer sheath 1200 that may be used in conjunction with an example tunneling device in accordance with the present disclosure. During a procedure to gain sub-sternal access and create a sub-sternal tunnel in a patient, e.g., to implant a medical device such as a lead 16, a tunneling shaft may be inserted into the inner lumen of introducer sheath 1200, e.g., wherein sheath 1200 is sized to extend from approximately a distal end to approximately a proximal end of the tunneling shaft prior to insertion and advancement of the tunneling shaft in a patient. Sheath 1200 includes a body 1202 and a handle 1204. Body 1202 of sheath 1200 defines an inner channel. In one example, sheath 1200 may be an open sheath as illustrated and described in U.S. patent application Ser. No. 14/196,298 and U.S. patent application Ser. No. 14/196,443, both of which are incorporated herein by reference in their entireties. In the case of an open sheath, sheath 1200 may include an opening along the length of body 1202 and the inner channel is accessible via the opening anywhere along the length of body 1202. In another example, sheath 1200 may be a splittable sheath in which body 1202 includes a score or other weakened portion to permit splitting of body 1202, e.g., as illustrated and described in further detail in U.S. patent application Ser. No. 14/196,443, previously incorporated above. In yet another example, sheath 1200 may be a sheath without any gap or score on body 1202, in which case sheath 1200 may be removed by slitting the sheath using a slitter, as illustrated and described in U.S. patent application Ser. No. 14/196,443, previously incorporated above. Sheath 1200 may have other properties describe above in reference to U.S. patent application Ser. No. 14/196,298 and U.S. patent application Ser. No. 14/196,443 or any commercially available sheaths.

The distal portion of the introducer sheath 1200 may have an open end so as to no obstruct light emitted by one or more lighting elements on a distal end of a tunneling shaft inside the lumen defined by sheath 1200. Also, the open end allows for insertion of a lead to the targeted area. Once a tunneling shaft is inserted into introducer sheath 1200, a distal end of the tunneling shaft may be inserted into an incision site, and then tunneled superiorly to both create a passageway and then form a sub-sternal tunnel. During the tunneling, a surgeon or other operator may control the advancement and direction of tunneling shaft 1200 by gripping handle 1204, which is located external to the body of patient. The surgeon or other operator may view the path of the tunneling shaft and sheath within the patient using an endoscope or other viewing device inserted inside the patient (e.g., in a pleural cavity of the patient). The surgeon or other operator may tunnel through patient tissue by way of blunt dissection using a distal end of the tunneling shaft. Then, for example, the tunneling shaft may be withdrawn from the patient's body, leaving the introducer sheath 1200 within the sub-sternal tunnel. The operator may pass a medical device, such as the above described lead 16, through the sheath lumen, via a proximal opening of the introducer sheath. The surgeon or other operator may then remove the introducer sheath 1200 from the body, leaving the lead within the sub-sternal tunnel, and then remove the sheath from the lead for example, by slitting or splitting the introducer sheath from around lead, according to some embodiments and methods.

In some examples, sheath 1200 may be at least partially translucent or transparent in order to allow light from lighting elements attached to a tunneling shaft inside the lumen of sheath 1200 to be seen outside of sheath 1200. While some of the examples above describe lighting elements disposed on or within a tunneling shaft, in some examples lighting element 1224 may be disposed on or within sheath 1200 that surrounds the tunneling shaft. For example, description above with reference to the location and attachment of lighting elements to a tunneling shaft may apply equally to location and attachment of lighting elements to sheath 1200. For example, lighting element 1224 may comprise a fiber optic array encircling a distal end of sheath body 1202. In some examples, lighting element 1224 may be disposed inside a lumen defined by sheath body 1202, and a distal end of sheath body 1202 may include a translucent or transparent window through which light emitted by lighting element 1224 may be seen outside of the lumen.

FIGS. 13A-13C are conceptual diagrams illustrating example arrangements of an example lighting element 1314 with respect to an example introducer sheath 1318 and example tunneling shaft 1312 in accordance with the present disclosure. While lighting elements according to some of the examples above are shown disposed on a tunneling shaft, in some examples lighting element 1314 may be disposed on a sheath that surrounds a tunneling shaft.

In the example of FIG. 13A, lighting element 1314 is disposed within a shaft lumen 1316 defined by tunneling shaft 1312. One or more of tunneling shaft 1312 and/or sheath 1318 may be at least partially translucent or transparent at the location along a length of the tunneling shaft 1312 and sheath 1318 where lighting element 1314 is disposed. In some examples, one or more of tunneling shaft 1312 and/or sheath 1318 may include a window disposed at the location to allow light emitted by lighting element 1314 to be seen outside of sheath 1318. In some examples, lighting element 1314 may be on a fiber disposed within shaft lumen 1316. In the example of FIG. 13B, lighting element 1314 is disposed outside shaft lumen 1316 and inside sheath lumen 1315. Lighting element 1314 may be attached to an exterior of tunneling shaft 1312, an interior of sheath 1318, or simply inserted in sheath lumen 1315. Sheath 1318 may include a window 1320 located along a length of sheath 1318 where lighting element 1314 is disposed to allow light emitted by lighting element 1314 to be seen outside of sheath 1318. In some examples, window 1320 may be covered by an at least partially translucent or transparent material (e.g., epoxy) to prevent body fluid from entering sheath 1318 while allowing light emitted by lighting element 1314 to be seen outside of sheath 1318.

In the example of FIG. 13C, lighting element 1314 is disposed outside sheath 1318. Lighting element 1314 may be attached to an exterior of sheath 1318.

In some examples, lighting element 1314 may be incorporated into the tunneling shaft (e.g., tunneling shaft 1312). For example, lighting element 1314 may be attached to an exterior or interior of tunneling shaft 1312 by epoxy or another appropriate method. In some examples, lighting element 1314 may be incorporated into the sheath (e.g., sheath 1318). For example, lighting element 1314 may be attached to an exterior or interior of sheath 1318 by epoxy or another appropriate method.

Although the description of FIGS. 13A-13C sometimes refer to sheath 1318, description of the placement of lighting element 1314 with respect to tunneling shaft 1312 may apply equally to examples of the present disclosure that do not include a sheath.

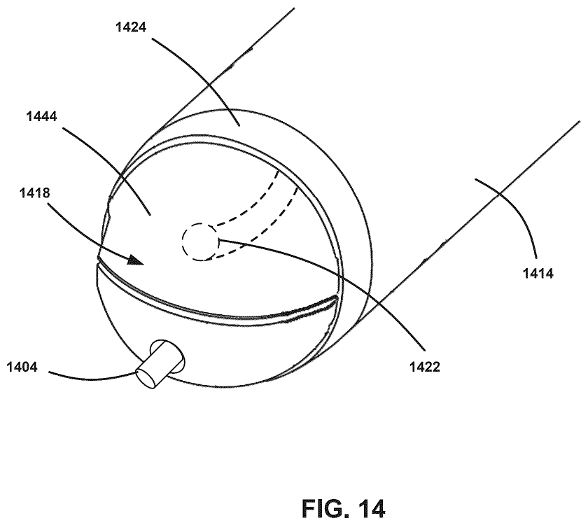

FIG. 14 is a conceptual diagram of an example distal portion of an example tunneling shaft 1414 in accordance with the present disclosure. The distal portion of tunneling shaft 1414 includes lighting element 1424, an optical window 1444, and a sensor pin 1404 on a distal end 1418 of the tunneling shaft 1414. Optical window 1444 may be formed of a transparent material, for example glass, quartz or clear plastics like polycarbonate (e.g., LEXAN) or acrylic. Optical window 1444 may allow optical sensor 1422 to sense light from outside the distal end 1418 of tunneling shaft 1414. Tunneling shaft 1414 may be part of a tunneling tool.

In some examples, the tunneling tool may include a pressure sensor to sense a pressure inside a patient. In some examples, the pressure sensor may be configured to measure a force pressure with which part of the tunneling tool is pressing against patient tissue. For example, the tunneling tool may include pin 1404 extending from tunneling shaft 14fnd resisting depression via a spring within tunneling shaft 1414. Although pin 1404 is depicted in FIG. 14 disposed at distal end 1418 of tunneling shaft 1414, in some examples pin 1404 may be disposed at any location along shaft 1414 that is inserted into the patient. As the tunneling tool is used to tunnel through patient tissue, some patient tissue may exert a force on pin 1404. A sufficient force acting on pin 1404 from the patient's tissue may overcome the force of the spring and depress pin 1404.

In some examples, a pressure sensor may be configured to measure a blood pressure of the patient. Lighting element 1424 may be configured to emit light at a particular wavelength and intensity to illuminate patient tissue surrounding distal end 1418. Optical sensor 1422 may be configured to sense light reflected off patient tissue and through optical window 1444. Optical window 1444 may be generally bulbous and define a blunt tip configured to facilitate dissection by dissecting tissue is an atraumatic manner. Based on changes in sensed light, processing circuitry (e.g., control circuitry 517 of FIG. 5) connected to optical sensor 1422 may be able to estimate a blood pressure of the patient. In some examples, optical sensor 1422 may be configured to view a path of tunneling shaft 1414 as it progresses through patient tissue.

Lighting element 1424 may be configured to change an operation state in response to a sensed pressure satisfying a threshold. For example, in response to sensing that a force pressure at the distal end 1418 exceeds a threshold pressure, control circuitry of the tunneling tool may cause lighting element 1424 to emit light in a fast flashing state and/or a red color state. In some examples, in response to sensing that a blood pressure of the patient exceeds a threshold blood pressure, control circuitry of the tunneling tool may cause lighting elements 1424 to emit light in varying flashing states and in varying color states. In some examples, control circuitry of the tunneling tool may change operation states of lighting element 1424 in response to pressure in any of the ways described above with respect to FIG. 5. In this way, an operator of the tunneling tool may quickly recognize potentially adverse conditions during an implant procedure.

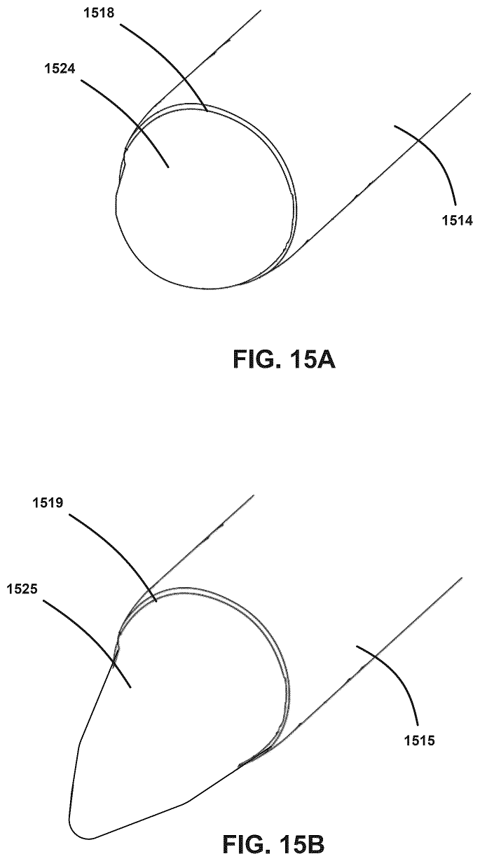

FIGS. 15A and 15B are conceptual diagrams of example distal portions of an example tunneling shaft in accordance with the present disclosure.

FIG. 15A shows tunneling shaft 1514 with lighting element 1524. In the example of FIG. 15A, lighting element 1524 is inserted into a lumen defined by tunneling shaft 1514 and progressed through the lumen until lighting element 1524 extends from distal end 1518 of tunneling shaft 1514. For example, tunneling shaft 1514 may include an endoscope with lighting element 1524 passed down a central lumen of the endoscope. In some examples, lighting element 1524 may comprise a fiberoptic component disposed near distal end 1518 of tunneling shaft 1514.

FIG. 15B shows tunneling shaft 1515 with lighting element 1525. In the example of FIG. 15B, lighting element 1525 is formed on distal end 1519 of tunneling shaft 1515. For example, lighting element 1525 may comprise an acrylic LED shape modified on distal end 1519 of tunneling shaft 1515.

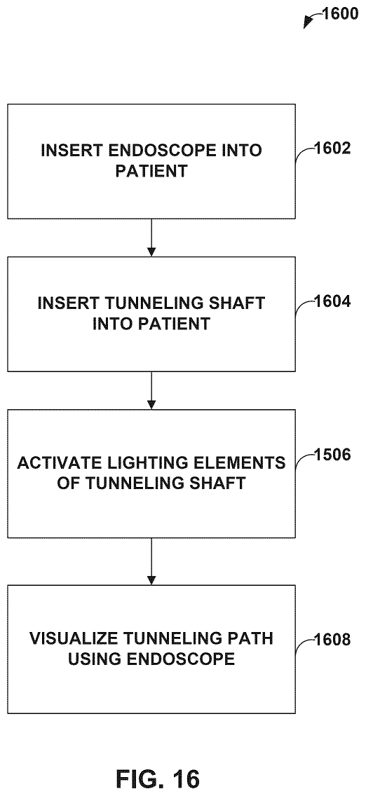

FIG. 16 is a flow diagram of an example technique for operating an implant system in accordance with the present disclosure. Method 1600 may be described with reference to FIGS. 1-5 above.

In operation according to one example, method 1600 includes inserting an endoscope into an internal cavity of a patient (1602). In some examples, inserting the endoscope into the patient includes inserting the endoscope into a pleural cavity of the patient. For example, an endoscopic incision 524 may be made in an intercostal space of patient 12, and a lung of patient 12 may be deflated (e.g., a left lung). As shown in FIG. 5, a shaft of endoscope 520 may be inserted into patient 12 through endoscopic incision 524, and be disposed within the left pleural cavity 35 of patient 12. A distal end of the endoscope shaft includes optical device 522 directed at a location of the anticipated tunneling trajectory for tunneling tool 510. Optical device 522 may be configured to sense light from lighting elements 524 during the implant procedure. In some examples, optical device 522 may be a camera configured to sense light at a variety of wavelengths and frequencies. Endoscope 520 may include processing circuitry (not shown) for receiving sensed signals from optical device 522 and sending signal data from the sensed signals to computing device 526. Computing device 526 may include processing circuitry configured to process the signal data into images viewable on a display of computing device 526 for an operator to visualize the interior of patient 12.

In some examples, inserting the endoscope into the patient includes inserting the endoscope into a substernal space of the patient. In some examples, the substernal space in which the endoscope is inserted is a space within transverse thoracic muscles of the patient. In some examples, the endoscope and tunneling shaft 514 may be advanced in a parallel fashion. For example, both the endoscope and tunneling shaft 514 of tunneling tool 510 may be inserted sub-xiphoid and advanced parallel to one another within patient 12. For example, tunneling tool 510 may be directly visible by a camera of the endoscope. In some examples, both the endoscope and tunneling shaft 514 may be advanced together. In some examples, the endoscope may be advanced separately from tunneling shaft 514.

In operation according to one example, method 1600 includes inserting a tunneling shaft 514 of tunneling tool 510 into a patient proximate the sternum (or some other part of the patient's body) (1605). A distal portion of tunneling shaft 514 may be inserted through incision site 504, e.g., at access site A shown in FIG. 2, with the user controlling the movement of shaft 514 by gripping handle 516, which is located externally. Handle 516 remains outside patient 12 to allow for a surgeon or other operator to maneuver tunneling shaft 514 along the desired path within the substernal space of patient 12. The user may manually orient distal end 518 beneath the skin. In some examples, tunneling tool 510 may include a guide member. The user may maintain the guide member outside the skin above the sternum.

In operation according to one example, method 1600 includes activating one or more lighting elements 524 of tunneling shaft 514 (1606). In some examples, actuation of actuation element 515 may cause control circuitry 517 to activate the one or more lighting elements 524. When activated, lighting elements 524 may be in an ON-state and emitting light according to one or more operation states. In some examples, control circuitry 517 may be configured to change operation states of lighting elements 514 in response to actuation of actuation element 515. For example, after a first actuation of actuation element 515 activates one or more of lighting elements 514, successive actuations of actuation element 515 may change the operation state of one or more of lighting elements 514, by way of control circuitry 517. Although described above with respect to a single actuation element 515, in some examples tunneling tool 510 includes a plurality of actuation elements, wherein each actuation element controls either a different lighting element of lighting elements 524 or a different operation state of one or more lighting elements of lighting elements 524. The one or more actuation elements may, by way of control circuitry 517, control lighting elements 524 to emit light at different wavelengths, intensities, and/or flashing intervals.

When tunneling tool 510 is inserted into patient 12 and lighting elements 524 are activated, the operator may be able to visualize the placement and trajectory of tunneling tool 510 within patient 12, as well as internal body structures of patient 12 (1608). When describing the operator visualizing features of tunneling tool 516 inside patient 12, it may be understood that the operator is viewing the features on a display of computing device 526 based on visualization of light emitted by one or more of lighting elements 514, e.g., as sensed data from optical device 522 (e.g., a camera) of endoscope 520.

The user may manipulate (e.g., rotate, push, pull, etc.) handle 516 as needed to perform dissection while tunneling into patient 12. For example, the user may push tunneling tool 510 distally to dissect tissue. In some examples, tunneling tool 510 may also include one or more electrodes and or sharp edges to break stubborn tissue areas and scar tissue. The user may also activate an electrode to cauterize tissue. The user may visualize the dissection path during operation of tunneling tool 510 using lighting elements 524 as seen by optical device 522. The user may externally visualize the internal path of dissection of tunneling tool 510 by looking at a display connected to optical device 522. Lighting elements 514 may represent a location of shaft 514 within patient 12, and may also illuminate patient tissue structures (e.g., a cardiac silhouette, adhesions in tissue, arteries, and veins), bleeding spots, or other conditions inside the patient during the implant procedure. Additionally, the user may refer to a guide member, which is moving along or above an outer surface of the sternum of the patient in an extracorporeal fashion and in a substantially parallel fashion to tunneling shaft 514.

The following examples are illustrative of the techniques described herein.

Example 1. A tunneling tool including: a handle; a tunneling shaft extending from the handle and defining a length; one or more lighting elements disposed along the length of the tunneling shaft, each of the one or more lighting elements configured to emit light detectable through tissue of a patient by an optical sensor.

Example 2. The tunneling tool of example 1, wherein the one or more lighting elements includes a lighting element disposed on a distal end of the tunneling shaft.

Example 3. The tunneling tool of example 1 or 2, wherein tunneling shaft includes a bend, and wherein the one or more lighting elements includes a lighting element disposed at an apex of the bend.

Example 4. The tunneling tool of any of examples 1-3, wherein the one or more lighting elements include at least two lighting elements disposed at different points along the length of the tunneling shaft.

Example 5. The tunneling tool of any of examples 1-4, wherein the one or more lighting elements include at least two lighting elements disposed at different points along the length of the tunneling shaft, and wherein each of the one or more lighting elements is configured to emit light at a different wavelength.

Example 6. The tunneling tool of any of examples 1-5, further including control circuitry configured to cause the one or more lighting elements to flash at predetermined time intervals.

Example 7. The tunneling tool of any of examples 1-6, further including control circuitry, wherein the one or more lighting elements include at least two lighting elements disposed at different points along the length of the tunneling shaft, and wherein the control circuitry is configured to cause each of the one or more lighting elements to flash at different time intervals.

Example 8. The tunneling tool of any of examples 1-7, further including control circuitry, wherein the control circuitry is configured to cause the one or more lighting elements to change an operation state in response to a predefined criteria being met.

Example 9. The tunneling tool of example 8, wherein the control circuitry is configured to implement one or more of the following operation state changes in the one or more lighting elements in response to the predetermined criteria being met: flash at a different time interval; emit light at a different wavelength; or emit light at a different intensity.

Example 10. The tunneling tool of any of examples 1-9, further including: a pressure sensor configured to sense a pressure inside the patient; and control circuitry, wherein the control circuitry is configured to cause the one or more lighting elements to change an operation state in response to the sensed pressure satisfying a threshold.

Example 11. The tunneling tool of example 10, wherein the control circuitry is configured to implement one or more of the following operation state changes in the one or more lighting elements in response to the sensed pressure satisfying the threshold: flash at a different time interval; emit light at a different wavelength; or emit light at a different intensity.

Example 12. The tunneling tool of any of examples 1-11, further including: an impedance sensor configured to sense an impedance in the patient's tissue; and control circuitry, wherein the control circuitry is configured to cause the one or more lighting elements to change an operation state in response to the sensed impedance satisfying a threshold.

Example 13. The tunneling tool of example 12, wherein the control circuitry is configured to implement one or more of the following operation state changes in the one or more lighting elements in response to the sensed impedance satisfying the threshold: flash at a different time interval; emit light at a different wavelength; or emit light at a different intensity.

Example 14. The tunneling tool of any of examples 1-13, further including: one or more activation elements; and control circuitry, wherein actuation of the one or more activation elements causes control circuitry to activate the one or more lighting elements.

Example 15. The tunneling tool of example 14, wherein the one or more activation elements are positioned on the handle for actuation by one or more of a user's fingers when the user grips the handle.

Example 16. The tunneling tool of example 14 or 15, wherein the actuation of the one or more activation elements causes control circuitry to change an operation state of the one or more lighting elements.

Example 17. The tunneling tool of any of examples 1-16, wherein the tunneling shaft extends in a curved orientation from a proximal end of the tunneling shaft to a distal end of the tunneling shaft.

Example 18. The tunneling tool of any of examples 1-17, further including a guide member extending from the handle that is adjacent to and coplanar with the tunneling shaft.

Example 19. The tunneling tool of example 18, wherein the guide member includes a distal end that is curved away from a sternum during use of the tunneling tool.

Example 20. The tunneling tool of any of examples 1-19, wherein the one or more lighting elements include a discrete light component.

Example 21. The tunneling tool of example 20 wherein the discrete light component is incorporated into the tunneling shaft.

Example 22. The tunneling tool of example 20, wherein the discrete light component is on a fiber within a lumen defined by the tunneling shaft.

Example 23. The tunneling tool of any of examples 1-22, wherein the one or more lighting elements include a fiberoptic component.

Example 24. The tunneling tool of example 23 wherein the fiberoptic component is disposed near a distal end of the tunneling shaft.

Example 25. The tunneling tool of any of examples 1-24, wherein the one or more lighting elements include a laser.

Example 26. The tunneling tool of any of examples 1-19, wherein the one or more lighting elements include a continuous linear light source along at least a portion of the length.

Example 27. The tunneling tool of example 26 wherein the continuous linear light source is disposed along a distal portion of the tunneling shaft.

Example 28. The tunneling tool of any of examples 1-21 or 22-27, wherein the one or more lighting elements encircle the tunneling shaft.

Example 29. The tunneling tool of any of examples 1-28, wherein the one or more lighting elements include a lighting element configured to emit light in a distinct, radial direction away from the tunneling shaft.

Example 30. An implant system including: a tunneling tool including: a handle including an activation element; and a tunneling shaft extending from the handle and defining a length; a plurality of lighting elements disposed along the length of the tunneling shaft, wherein each of the plurality of lighting elements is configured to emit light at a different wavelength or flash light at different time intervals; and an endoscope including a camera configured to visualize a dissection path of the tunneling tool through visualization of light emitted by the plurality of lighting elements through tissue of the patient using the camera.

Example 31. The implant system of example 30, wherein the one or more lighting elements includes a lighting element disposed on a distal end of the tunneling shaft.

Example 32. The implant system of example 30 or 31, wherein tunneling shaft includes a bend, and wherein the one or more lighting elements includes a lighting element disposed at an apex of the bend.

Example 33. The implant system of any of examples 30-32, wherein the one or more lighting elements include at least two lighting elements disposed at different points along the length of the tunneling shaft.

Example 34. The implant system of any of examples 30-33, wherein the one or more lighting elements include at least two lighting elements disposed at different points along the length of the tunneling shaft, and wherein each of the one or more lighting elements is configured to emit light at a different wavelength.

Example 35. The implant system of any of examples 30-34, further including control circuitry configured to cause the one or more lighting elements to flash at predetermined time intervals.

Example 36. The implant system of any of examples 30-35, further including control circuitry, wherein the one or more lighting elements include at least two lighting elements disposed at different points along the length of the tunneling shaft, and wherein the control circuitry is configured to cause each of the one or more lighting elements to flash at different time intervals.

Example 37. The implant system of any of examples 30-36, further including control circuitry, wherein the control circuitry is configured to cause the one or more lighting elements to change an operation state in response to a predefined criteria being met.

Example 38. The implant system of example 30-37, wherein the control circuitry is configured to implement one or more of the following operation state changes in the one or more lighting elements in response to the predetermined criteria being met: flash at a different time interval; emit light at a different wavelength; or emit light at a different intensity.

Example 39. The implant system of any of examples 30-38, further including: one or more activation elements; and control circuitry, wherein actuation of the one or more activation elements causes control circuitry to activate the one or more lighting elements.

Example 40. The implant system of example 39, wherein the actuation of the one or more activation elements causes control circuitry to change an operation state of the one or more lighting elements.

Example 41. The implant system of example 39 or 40, wherein the one or more activation elements are positioned on the handle for actuation by one or more of a user's fingers when the user grips the handle.

Example 42. The implant system of example 39 or 40, further including a remote, wherein the one or more activation elements are positioned on the remote handle for actuation by one or more of a user's fingers when the user grips the remote.

Example 43. The implant system of any of examples 30-42, wherein the tunneling shaft extends in a curved orientation from a proximal end of the tunneling shaft to a distal end of the tunneling shaft.

Example 44. The implant system of any of examples 30-43, further including a guide member extending from the handle in a substantially parallel fashion to the tunneling shaft, the guide member configured to move above an outer surface of a sternum in an extracorporeal fashion during use of the tunneling tool.

Example 45. The implant system of example 44, wherein the guide member includes a distal end that is curved away from a sternum during use of the tunneling tool.

Example 46. The implant system of any of examples 30-45, wherein the one or more lighting elements include a discrete light component.

Example 47. The implant system of any of examples 30-45, wherein the one or more lighting elements include a continuous linear light source along at least a portion of the length.

Example 48. The implant system of any of examples 30-47, wherein the one or more lighting elements include a fiberoptic component.

Example 49. The implant system of any of examples 30-48, wherein the one or more lighting elements include a laser.

Example 50. The implant system of any of examples 30-49, wherein the one or more lighting elements include a lighting element configured to emit light in a distinct, radial direction away from the tunneling shaft.

Example 51. The implant system of any of examples 30-50, wherein the one or more lighting elements are disposed within a lumen defined by the tunneling shaft.

Example 52. The implant system of any of examples 30-51, wherein the one or more lighting elements are incorporated into the tunneling shaft.

Example 53. The implant system of any of examples 30-51, further including a sheath defining a sheath lumen, wherein the tunneling shaft is configured to be disposed within the sheath lumen, and wherein the one or more lighting elements are incorporated into the sheath.

Example 54. The implant system of any of examples 30-53, further including a display and processing circuitry configured to: output an image to the display based on sensed data from the camera.

Example 55. The implant system of example 54, wherein the processing circuitry is further configured to estimate a trajectory the tunneling shaft and project the trajectory onto the display.

Example 56. The implant system of example 54 or 55, wherein the processing circuitry is further configured to: identify key tissues within the patient; determine a distance of the tunneling tool from the key tissues; and cause the one or more lighting elements to change an operation state in response to the distance satisfying a threshold.

Example 57. A method including: inserting an endoscope into a patient, wherein the endoscope includes a camera; inserting a tunneling shaft of a tunneling tool into a substernal space of the patient, wherein the tunneling tool includes: a handle; the tunneling shaft extending from the handle and defining a length; one or more lighting elements disposed along the length of the tunneling shaft; and control circuitry; actuating an activation element to cause the control circuitry to activate the one or more lighting elements; and visualizing a dissection path of the tunneling tool through visualization of light emitted by the one or more lighting elements using the camera.

Example 58. The method of example 57, wherein inserting the endoscope into the patient includes inserting the endoscope into a pleural cavity of the patient.

Example 59. The method of example 57, wherein inserting the endoscope into the patient includes inserting the endoscope into the substernal space of the patient, wherein the method further includes advancing the endoscope and tunneling shaft in a parallel fashion.

Example 60. The method of any of examples 57-59, wherein the substernal space includes a space within transverse thoracic muscles of the patient.

Various examples have been described. These and other examples are within the scope of the following claims.

What is claimed is:

1. A tunneling tool comprising:
a handle;
a tunneling shaft extending from the handle and defining a length;
one or more lighting elements disposed along the length of the tunneling shaft, each of the one or more lighting elements configured to emit light detectable through tissue of a patient by an optical sensor.

2. The tunneling tool of claim 1, wherein the one or more lighting elements comprise at least two lighting elements disposed at different points along the length of the tunneling shaft.

3. The tunneling tool of claim 2, further comprising control circuitry, and wherein the control circuitry is configured to cause each of the one or more lighting elements to one or more of:
flash at different time intervals;
emit light at a different wavelength; and
emit light at a different intensity

4. The tunneling tool of claim 1, further comprising control circuitry, wherein the control circuitry is configured to cause the one or more lighting elements to change an operation state in response to a predefined criteria being met, wherein the operation states include one or more of: flash at a different time interval; emit light at a different wavelength; or emit light at a different intensity.

5. The tunneling tool of claim 4, wherein the predefined criteria include one or more of:
a distance between the tunneling tool from and key tissues satisfying a threshold;
a sensed pressure inside the patient satisfying a threshold; and
a sensed impedance of patient tissue satisfying a threshold.

6. The tunneling tool of claim 1, further comprising:
one or more activation elements; and
control circuitry, wherein the actuation of the one or more activation elements causes the control circuitry to change an operation state of the one or more lighting elements.

7. The tunneling tool of claim 1, wherein the one or more lighting elements comprise one or more of:
a discrete light component;
a continuous linear light source;
a fiberoptic component;
a laser; and
an LED.

8. The tunneling tool of claim 1, wherein the one or more lighting elements are incorporated into the tunneling shaft.

9. The tunneling tool of claim 1, wherein the one or more lighting elements encircle the tunneling shaft.

10. The tunneling tool of claim 1, wherein the one or more lighting elements comprise a lighting element configured to emit light in a distinct, radial direction away from the tunneling shaft.

11. The tunneling tool of claim 1, wherein the one or more lighting elements are disposed on a fiber within a lumen defined by the tunneling shaft.

12. An implant system comprising:
a tunneling tool comprising:
a handle comprising an activation element; and
a tunneling shaft extending from the handle and defining a length;
a plurality of lighting elements disposed along the length of the tunneling shaft, wherein each of the plurality of lighting elements is configured to emit light at a different wavelength or flash light at different time intervals; and
an endoscope comprising a camera configured to visualize a dissection path of the tunneling tool through visualization of light emitted by the plurality of lighting elements through tissue of the patient using the camera.

13. The tunneling tool of claim 12, further comprising control circuitry,
wherein the one or more lighting elements comprise at least two lighting elements disposed at different points along the length of the tunneling shaft, and
wherein the control circuitry is configured to cause each of the one or more lighting elements to one or more of:
flash at different time intervals;
emit light at a different wavelength; and
emit light at a different intensity

14. The implant system of claim 12, further comprising a sheath defining a sheath lumen, wherein the tunneling shaft is configured to be disposed within the sheath lumen, and wherein the one or more lighting elements are incorporated into the sheath.

15. The implant system of claim 12, further comprising a display and processing circuitry configured to:
output an image to the display based on sensed data from the camera;
estimate a trajectory of the tunneling shaft; and
project the trajectory onto the display.

16. The tunneling tool of claim 12, further comprising control circuitry, wherein the control circuitry is configured to cause the one or more lighting elements to change an operation state in response to a predefined criteria being met, wherein the operation states include one or more of:
flash at a different time interval; emit light at a different wavelength; or emit light at a different intensity.

17. The tunneling tool of claim 16, wherein the predefined criteria include one or more of:
a distance between the tunneling tool from and key tissues satisfying a threshold;
a sensed pressure inside the patient satisfying a threshold; and
a sensed impedance of patient tissue satisfying a threshold.

18. A method comprising:
inserting an endoscope into a patient, wherein the endoscope includes a camera;
inserting a tunneling shaft of a tunneling tool into a substernal space of the patient, wherein the tunneling tool comprises:
a handle;
the tunneling shaft extending from the handle and defining a length;
one or more lighting elements disposed along the length of the tunneling shaft; and
control circuitry;
actuating an activation element to cause the control circuitry to activate the one or more lighting elements; and
visualizing a dissection path of the tunneling tool through visualization of light emitted by the one or more lighting elements using the camera.

19. The method of claim 18, wherein inserting the endoscope into the patient comprises inserting the endoscope into a pleural cavity of the patient.

20. The method of claim 18, wherein inserting the endoscope into the patient comprises inserting the endoscope into the substernal space of the patient, wherein the method further comprises advancing the endoscope and tunneling shaft in a parallel fashion.
